(19) United States
(12) Patent Application Publication
(10) Pub. No.: US 2005/0014789 A1
(43) Pub. Date: Jan. 20, 2005

(54) Amide derivatives as selective serotonin re-uptake inhibitors

(75) Inventors: Mark David Andrews, County of Kent (GB); Alan Daniel Brown, County of Kent (GB); David Sebastien Fradet, County of Kent (GB); David William Gordon, County of Kent (GB); Mark Ian Lansdell, County of Kent (GB); Malcolm Christian MacKenny, County of Kent (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/865,408

(22) Filed: Jun. 10, 2004

(30) Foreign Application Priority Data
Jun. 17, 2003 (GB) 0314054.8

(51) Int. Cl. A61K 31/445; A61K 31/40; C07D 211/56; C07D 207/10

(52) U.S. Cl. 514/317; 514/426; 546/224; 548/557

(57) ABSTRACT

The present invention relates to compounds of formula (I), 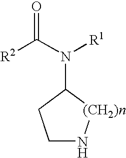
wherein R¹ is selected from:
(a) (C₁-C₆)alkyl, optionally substituted by 1-3 substituents, each independently selected from:
   (i) CF₃, OH, (C₁-C₆)alkyl, (C₃-C₆)cycloalkyl, (C₁-C₆)alkoxy and halo.
   (ii) Phenyl, optionally fused with phenyl or cyclohexyl, said phenyl or fused phenyl optionally substituted with 1-3 groups selected from (C₁-C₆)alkyl, (C₁-C₆)alkyl ester, OH and halo; and
(b) (C₃-C₆)cycloalkyl, optionally fused with (C₅-C₇)cycloalkyl, said cycloalkyl or fused cycloalkyl optionally substituted by OH, (C₁-C₆)alkyl, (C₁-C₆)alkoxy and halo.

R² is Phenyl, optionally fused to (C₄-C₆)cycloalkyl, phenyl or pyridyl, said phenyl or fused phenyl moiety optionally substituted with 1-3 groups each independently selected from (C₁-C₆)alkyl, (C₁-C₆)alkoxy, halo and OH; n is 1 to 2; and pharmaceutically acceptable salts, solvates or polymorphs thereof;

With the proviso that when n is 2 and R¹ is 2-(3,4-dimethoxylphenyl)-1-ethyl, 3,3-diphenyl-1-propyl or 2,4-difluorophenyl, then R² cannot be 4-trifluoromethoxyphenyl, 2,4,6-trimethoxyphenyl, 4-acetoxyphenyl or 2,4-difluorophenyl; which are a class of selective serotonin re-uptake inhibitors (SSRIs).

### CROSS-REFERENCE TO RELATED APPLICATION

This is a non-provisional patent application, which claims priority from United Kingdom Application Number 0314054.8, filed on Jun. 17, 2003.

This invention relates to novel compounds which inhibit monoamine re-uptake. In particular compounds of the present invention exhibit activity as selective serotonin re-uptake inhibitors (SSRIs) and have utility therefore in a variety of therapeutic areas. Notably the compounds of the present invention are useful in the treatment or prevention of a variety of disorders, including those in which the regulation of monoamine transporter function is implicated, such as depression, attention deficit hyperactivity disorder, obsessive-compulsive disorder, post-traumatic stress disorder, substance abuse disorders and sexual dysfunction including premature ejaculation, and to pharmaceutical formulations containing such compounds.

According to a first aspect, the invention provides a compound of general formula (I), pharmaceutically acceptable salts, solvates or polymorphs thereof;

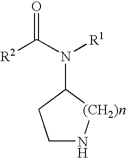

Wherein R¹ is selected from:
(a) (C₁-C₆)alkyl, optionally substituted by 1-3 substituents, each independently selected from:
   (i) CF₃, OH, (C₁-C₆)alkyl, (C₃-C₆)cycloalkyl, (C₁-C₆)alkoxy and halo.
   (ii) Phenyl, optionally fused with phenyl or cyclohexyl, said phenyl or fused phenyl optionally substituted with 1-3 groups selected from (C₁-C₆)alkyl, (C₁-C₆)alkyl ester, OH and halo; and
(b) (C₃-C₆)cycloalkyl, optionally fused with (C₅-C₇)cycloalkyl, said cycloalkyl or fused cycloalkyl optionally substituted by OH, (C₁-C₆)alkyl, (C₁-C₆)alkoxy and halo.

R² is Phenyl, optionally fused to (C₄-C₆)cycloalkyl, phenyl or pyridyl, said phenyl or fused phenyl moiety optionally substituted with 1-3 groups each independently selected from (C₁-C₆)alkyl, (C₁-C₆)alkoxy, halo and OH;
n is 1 or 2;
with the proviso that when n is 2 and R¹ is 2-(3,4-dimethoxylphenyl)-1-ethyl, 3,3-diphenyl-1-propyl or 2,4-difluorophenyl, then R2 cannot be 4-trifluoromethoxyphenyl, 2,4,6-trimethoxyphenyl, 4-acetoxyphenyl or 2,4-difluorophenyl.

Unless otherwise indicated, any alkyl group may be straight or branched.

Halo means fluoro, chloro, bromo or iodo.

Preferably R¹ is selected from:
(a) (C₁-C₆)alkyl, optionally substituted by 1-3 substituents, each independently selected from:
   (i) CF₃, OH, (C₁-C₆)alkyl, (C₃-C₆)cycloalkyl, (C₁-C₆)alkoxy and halo.
   (ii) Phenyl, optionally fused with phenyl or cyclohexyl, said phenyl or fused phenyl optionally substituted with 1-3 groups selected from (C₁-C₆)alkyl, (C₁-C₆)alkyl ester, OH and halo; and
(b) (C₃-C₆)cycloalkyl, optionally fused with (C₅-C₇)cycloalkyl.

More preferably R1 is selected from:
(a) (C₁-C₅)alkyl, optionally substituted by 1-2 substituents, each independently selected from:
   (i) OH, (C₃-C₄)cycloalkyl,.
   (ii) Phenyl optionally substituted by C(O)OCH₃
(b) (C₄-C₅)cycloalkyl

Yet more preferably R¹ is selected from: (C₃-C₅)alkyl; (C₁)alkyl substituted by phenyl, cyclobutyl or cyclopropyl; and (C₄-C₅)cycloalkyl.

Preferably R² is phenyl, optionally fused to cyclohexyl, phenyl or pyridyl, said phenyl or fused phenyl moiety optionally substituted with 1-3 groups each independently selected from methoxy, methyl, chloro and fluoro. More preferably R² is phenyl, optionally fused to cyclohexyl, phenyl or pyridyl, said phenyl or fused phenyl moiety optionally substituted with 1-3 groups each independently selected from methoxy, methyl and chloro.

More preferably still R² is phenyl, optionally substituted with 2-3 groups each independently selected from methoxy, methyl and chloro; or phenyl fused to cyclohexyl, phenyl or pyridyl and optionally substituted with 1-2 groups independently selected from methyl, methoxy and chloro.

Preferably n is 1.

Preferred compounds are:
N-Cyclopentyl-N-piperidin-4-yl-2-naphthamide;
N-Butyl-3-chloro-2-methyl-N-piperidin-4-ylbenzamide;
3-Chloro-N-(cyclopropylmethyl)-2-methyl-N-piperidin-4-ylbenzamide;
N-Isobutyl-N-[(3R)-pyrrolidin-3-yl]-2-naphthamide;
N-Isobutyl-N-[(3R)-pyrrolidin-3-yl]quinoline-6-carboxamide;
N-Isobutyl-N-[(3S)-pyrrolidin-3-yl]quinoline-6-carboxamide;
N-(3-Methylbutyl)-N-[(3R)-pyrrolidin-3-yl]-2-naphthamide;
3,4-Dichloro-N-isopropyl-2-methyl-N-[(3R)-pyrrolidin-3-yl]benzamide;
N-Butyl-1 -methyl-N-[(3R)-pyrrolidin-3-yl]-5,6,7,8 tetrahydronaphthalene-2-carboxamide
N-Butyl-N-[(3R)-pyrrolidin-3-yl]-2-naphthamide
N-Butyl-1-chloro-N-[(3R)-pyrrolidin-3-yl]-2-naphthamide
N-Isobutyl-3-methoxy-2-methyl-N-[(3R)-pyrrolidin-3-yl]benzamide

Compounds of formula (I) encompasses compounds of formula (IA) and (IB) as racemic mixtures and as single enantiomers.

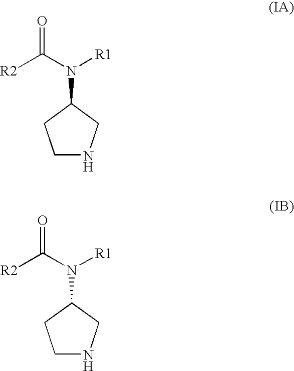

Particularly preferred are compounds of formula (IA).

For the avoidance of doubt, unless otherwise indicated, the term substituted means substituted by one or more defined groups. In the case where groups may be selected from a number of alternatives groups, the selected groups may be the same or different.

For the avoidance of doubt, the term independently means that where more than one substituent is selected from a number of possible substituents, those substituents may be the same or different.

The compounds of the invention have the advantage that they are selective inhibitors of the re-uptake of serotonin (SRIs) (and so are likely to have reduced side effects), they have a rapid onset of action (making them suitable for administration shortly before an effect is required), they have desirable potency and associated properties. Compounds that selectively inhibit the re-uptake of serotonin, but not noradrenaline or dopamine, are preferred.

The pharmaceutically or veterinarily acceptable salts of the compounds of formula I which contain a basic centre are, for example, non-toxic acid addition salts formed with inorganic acids such as hydrochloric, hydrobromic, hydroiodic, sulfuric and phosphoric acid, with carboxylic acids or with organo-sulfonic acids. Examples include the HCl, HBr, HI, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. Compounds of the invention can also provide pharmaceutically or veterinarily acceptable metal salts, in particular non-toxic alkali and alkaline earth metal salts, with bases. Examples include the sodium, potassium, aluminium, calcium, magnesium, zinc, diolamine, olamine, ethylenediamine, tromethamine, chloine, megulamine and diethanolamine salts. For reviews on suitable pharmaceutical salts see Berge et al, J. Pharm, Sci., 66, 1-19, 1977; P L Gould, International Journal of Pharmaceutics, 33 (1986), 201-217; and Bighley et al, Encyclopedia of Pharmaceutical Technology, Marcel Dekker Inc, New York 1996, Volume 13, page 453-497.

Hereinafter, the compounds, their pharmaceutically acceptable salts, their solvates and polymorphs, defined in any aspect of the invention (except intermediate compounds in chemical processes) are referred to as “compounds of the invention”.

The pharmaceutically acceptable solvates of the compounds of the invention include the hydrates thereof.

The compounds of the invention may possess one or more stereogenic centres and so exist in a number of stereoisomeric forms. All stereoisomers and mixtures thereof are included in the scope of the present invention. Racemic compounds may either be separated using preparative HPLC and a column with a chiral stationary phase or resolved to yield individual enantiomers utilising methods known to those skilled in the art. In addition, chiral intermediate compounds may be resolved and used to prepare chiral compounds of the invention.

The compounds of the invention may exist in one or more tautomeric forms. All tautomers and mixtures thereof are included in the scope of the present invention. For example, a claim to 2-hydroxypyridinyl would also cover its tautomeric form, α-pyridonyl. In cases where compounds of the invention exist as tautomeric isomers, the invention includes individual tautomers as well as mixtures thereof.

In cases where the compounds of the invention exist as optical isomers, the invention includes individual isomers as well as mixtures thereof. In cases where the compounds of the invention exist as diastereoisomers, the invention includes individual diastereoisomers as well as mixtures thereof.

It will be appreciated by those skilled in the art that certain protected derivatives of compounds of the invention, which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may act as prodrugs of other compounds of the invention.

All protected derivatives and prodrugs of compounds of the invention are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference).

It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within the compounds of the invention.

Preferred prodrugs for compounds of the invention include: esters, carbonate esters, hemi-esters, phosphate esters, nitro esters, sulfate esters, sulfoxides, amides, carbamates, azo-compounds, phosphamides, glycosides, ethers, acetals and ketals.

The invention also includes all suitable isotopic variations of the compounds of the invention. An isotopic variation is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the invention, for example, those in which a radioactive isotope such as ³H or ¹⁴C is incorporated, are useful in drug and/or substrate tissue distribution studies. Tritiated, i.e. ³H, and carbon-14, i.e. ¹⁴C isotopes are particularly preferred for their ease of preparation and detectability. Further, substitution with isotopes such as deuterium, i.e. ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the methods or preparations described in the Examples and Preparations hereafter using appropriate isotopic variations of suitable reagents.

Compounds of the invention may be prepared, in known manner in a variety of ways. In the following reaction schemes and hereafter, unless otherwise stated, R, R² and n are as defined in the first aspect. These processes form further aspects of the invention.

Throughout the specification, general formulae are designated by Roman numerals I, II, III, IV etc.

Compounds of formula (I), where R¹, R² and n are as herein described may be prepared from compounds of formula (II) and (III) as described below:

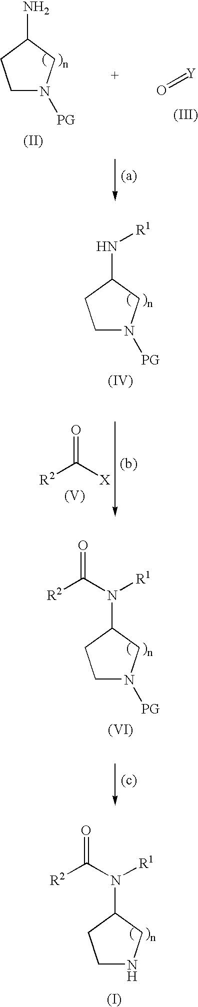

Compounds of formula (IV) may be prepared from compounds of formula (II) where PG is a nitrogen protecting group and (III) where Y—H is R¹, under the conditions of process step (a)—Reductive Amination; the dehydration of an amine and an aldehyde or ketone followed by the reduction of the resultant imine by a metal hydride reagent or hydrogenation, in a suitable solvent at room temperature.

Examples of suitable nitrogen protecting groups include benzyl, tert-butoxycarbonyl (BOC) and trifluoromethylacetamide. Particularly suitable are acid labile nitrogen protecting groups such as BOC. Such groups are well known to the man skilled in the art and are detailed in “Protecting Groups in Organic Synthesis”, 3rd edition, by T W Greene and P G M Wuts, John Wiley and Sons Inc, 1999.

Typical reaction conditions comprise treating equimolar amounts of amine and aldehyde or ketone with either sodium triacetoxyborohydride (STAB), NaCN(BH)₃ or NaBH₄, in a suitable solvent (e.g. dichloromethane (DCM), tetrahydrofuran (THF)) at room temperature for 1 to 24 hours. Alternatively, an excess of a reducing agent (e.g. NaBH₄, LiAlH₄, STAB) may be used in a suitable solvent (e.g. THF, MeOH, EtOH) after the amine and aldehyde or ketone have been mixed for 1-18 hours, optionally in the presence of a drying agent (e.g. molecular sieve) or using Dean-Stark apparatus with a suitable solvent (e.g. toluene, xylene) or by evaporation of a suitable solvent mixture (e.g. toluene and MeOH) under reduced pressure, to facilitate the removal of water. Alternatively still, reduction can be carried out by catalytic hydrogenation in the presence of a palladium or nickel catalyst (e.g. Pd/C, Raney® Ni) under an atmosphere of H₂, optionally at elevated temperature and pressure, in a suitable solvent. In certain cases it may be desirable to use an excess of the alsehyde or ketone of formula (III).

Preferably the aldehyde or ketone and the amine are treated with either 10% Pd/C, (optionally in the presence of triethylamine), in ethanol under 60psi of hydrogen at room temperature for 18 hours, or with sodium borohydride in methanol at room temperature for 1-16 hours.

Compounds of formula (VI) may be prepared by the reaction of compounds of formula (IV) with compounds of formula (V), where X is OH or Cl, under the conditions of process step (b)—Amide Formation.

Peptide linkage of compound (V) with amine (IV) may be performed by reaction of:
(i) an acyl chloride derivative of acid (V) (when X is Cl), with an amine (IV), in the presence of an excess of acid acceptor in a suitable solvent, or
(ii) an acid (V), (when X is OH), (optionally in the presence of a conventional coupling agent), with the amine (IV), and further, optionally in the presence of a catalyst, with an excess of acid acceptor in a suitable solvent.

Typically the reaction conditions are as follows:
(i) the acid chloride of acid (V) (optionally generated in-situ), is treated with amine (IV), optionally in the presence of an excess of a 30 amine such as Et₃N, Hünig's base or N-methyl morpholine (NMM), in DCM or dioxane, optionally at elevated temperature for 1 to 24 hrs, or
(ii) the acid (V), a coupling agent such as 1-(3-dimethylaminopropyl)-3-ethylcarbodiimide hydrochloride (WSCDI) or dicyclohexylcarbodiimide (DCC); in the presence of one of 1-hydroxybenzotriazole (HOBT) or 1-hydroxy-7-azabenzotriazole (HOAT); amine (IV), with an excess of an amine base such as NMM, Et₃N or Hünig's base in a suitable solvent such as THF, DCM or EtOAc, at rt. for 1 to 48 hrs; or alternatively:
   acid (V), a coupling agent such as O-(Benzotriazol-1-yl)-N,N,N′,N′-tetramethyluronium tetrafluoroborate (TBTU), PYBOP®, PyBrOP® or Mukaiyama's reagent, and an excess of amine (IV), with an amine base such as NMM, Et₃N or Hünig's base are combined in THF, DCM or EtOAc, at rt. for 4 to 24 hrs.

When X═OH, preferred conditions are treatment of acid (V) with oxalyl chloride and N,N-dimethylformamide in dichloromethane at room temperature for 1 hour followed by treatment with amine (IV) and triethylamine in DCM at rt for 18 hrs. When X═Cl, preferred conditions are treatment of acyl chloride (V) with amine (IV) and triethylamine in DCM at rt for 18 hrs.

Compounds of formula (I) may be prepared from compounds of formula (VI) under the conditions of process step \mathring{c}—Deprotection. Where PG is a suitable amine-protecting group, preferably BOC, trifluoroacetate or benzyl (Bz), removal of PG from amide (VI), to form unprotected amide (I) is achieved by a method selective to the protecting group as detailed in ‘Protective Groups in Organic Synthesis’, 3rd edition, by T W Greene and P G M Wuts, John Wiley and Sons Inc, 1999.

Typically:

When PG is BOC, deprotection is achieved by treatment of (VI) with an excess of strong acid (e.g. HCl, TFA) at room temperature in a suitable solvent (e.g. DCM, EtOAc, dioxan).

When PG is trifluoroactetate treatment of (VI) with a base (e.g. K₂CO₃, Na₂CO₃, NH₃, Ba(OH)₂) in an alcoholic solvent (e.g. MeOH, EtOH), optionally with water and optionally at elevated temperature.

When PG is Bz either transfer hydrogenation with a transition metal or transition metal salt hydrogenation catalyst (e.g. Pd/C, Pd(OH)₂) in the presence of a hydrogen donor (e.g. H₂, NH₄⁺HCO₂⁻) in a polar solvent (e.g. tetrahydrofuran, ethanol, methanol) optionally at elevated temperature and/or pressure. Or, alternatively by catalytic hydrogenation in the presence of a palladium or nickel catalyst (e.g. Pd/C, Raney® Ni) under an atmosphere of H₂, optionally at elevated temperature and pressure, in a suitable solvent. Preferably, when PG is BOC, deprotection is achieved by treatment with an excess of 4M hydrochloric acid in dioxan for 18 hours at room temperature.

When PG is trifluoroactetate, deprotection is achieved by treatment with K₂CO₃ in methanol and water (5:1 to 10:1 mixture) at room temperature for 18 hours.

When PG is Bz, deprotection is achieved by treatment with NH₄⁺ HCO₂⁻ and 10% Pd/C in ethanol under gentle reflux for between 6 and 20 hours.

Alternatively, compounds of formula (IV) where R¹ and n are as described herein may be prepared from compounds of formula (VII) and (VIII) as described below:

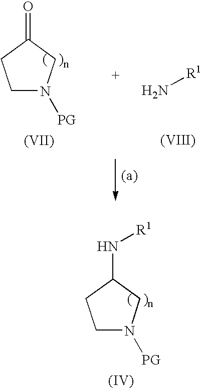

Compounds of formula (IV) may be prepared from compounds of formula (VII) where PG is a nitrogen protecting group, and (VIII), under the conditions of process step (a)—Reductive Amination, as described above.

Additionally, compounds of formula (IV) where R¹ and n are as described herein may be prepared from compounds of formula (II) as described below:

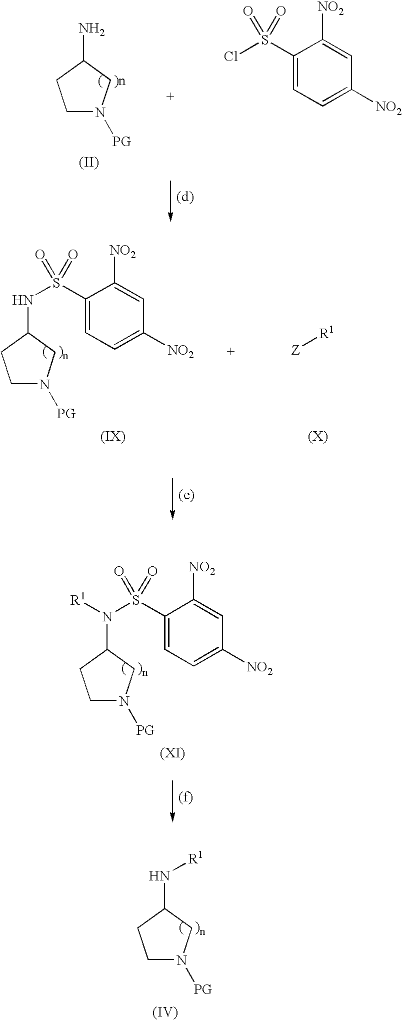

Compounds of formula (IX) may be prepared from compounds of formula (II) by sulfonylation with 2,4-dinitrosulfonyl chloride, under the conditions of process step (d)—Sulfonamide formation

Typical reaction conditions comprise treating equimolar amounts of amine (II) and 2,4-dinitrosulfonyl chloride with an excess of a suitable base, such as lutidine, in a suitable solvent, such as dichloromethane, at 0° C. to room temperature for 1 to 24 hrs

Compounds of formula (XI) may be prepared from compounds of formula (IX) by alkylation with a compound of formula (X), under the conditions of process step (e)—Sulfonamide alkylation

Alklyation of sulfonamides (IX) with compound of formula (X) may be performed by reaction of:
(i) an activated alkyl compound (X) (when Z is a suitable leaving group such as Cl, Br, I or a sulfonate ester such as methanesulfonate or p-toluenesulfonate), with a sulfonamide (IX), in the presence of a base in a suitable solvent, or
(ii) the alcohol (X), (when Z is OH), with a sulfonamide (IX) in the presence of a 3° phosphine and an azodicarboxylic acid diester or diamide in a suitable solvent.

Typically the reaction conditions are as follows:
(i) the sulfonamide (IX), is treated with an activated alkyl compound (X) (when Z is a suitable leaving group such as Cl, Br, I or a sulfonate ester such as methanesulfonate or p-toluenesulfonate), in the presence of an excess of a base such as K₂CO₃, Et₃N, Hünig's base or N-methyl morpholine (NMM), in DCM, CH₃CN or dioxane, optionally at elevated temperature for 1 to 24 hrs, or
(ii) the sulfonamide (IX) is treated with an alcohol (X) (Z=OH) in the presence of a 3° phosphine, such as triphenylphosphine and an azodicarboxylic acid diester or diamide, such as diisopropyl azodicarboxylate, in THF at 0° C. and then allowed to warm to room temperature and stirred for 30 min to 16 hrs.

Compounds of formula (IV) may be prepared from compounds of formula (XI) under the conditions of process step (f)—Sulfonamide cleavage Typical reaction conditions comprise treating the sulfonamide (XI) with an excess of a suitable nucleophile, such as mercaptoacetic acid, in the presence of a suitable base, such as triethylamine or Hunig's base, in a suitable solvent, such as dichloromethane, at room temperature for 30 min to 16 hrs

Compounds of formulae (II), (III), (V), (VII) and (VIII) are either known and available from commercial sources or are derivable from commercially available starting materials using techniques well known to those skilled in the art (see Examples hereinafter).

It will be apparent to those skilled in the art that sensitive functional groups may need to be protected and deprotected during synthesis of a compound of formula I. This may be achieved by conventional techniques, for example as described in ‘Protective Groups in Organic Synthesis’, 3^{rd} edition, by T W Greene and P G M Wuts, John Wiley and Sons Inc, 1999.

The compounds of the invention are useful because they have pharmacological activity in mammals, including humans. More particularly, they are useful in the treatment or prevention of a disorder in which the regulation of monoamine transporter function is implicated. Disease states that may be mentioned include hypertension, depression (e.g. depression in cancer patients, depression in Parkinson's patients, postmyocardial infarction -depression, subsyndromal symptomatic depression, depression in infertile women, paediatric depression, major depression, single episode depression, recurrent depression, child abuse induced depression, post partum depression and grumpy old man syndrome), generalized anxiety disorder, phobias (e.g. agoraphobia, social phobia and simple phobias), posttraumatic stress syndrome, avoidant personality disorder, premature ejaculation, eating disorders (e.g. anorexia nervosa and bulimia nervosa), obesity, chemical dependencies (e.g. addictions to alcohol, cocaine, heroin, phenobarbital, nicotine and benzodiazepines), cluster headache, migraine, pain, Alzheimer's disease, obsessive-compulsive disorder, panic disorder, memory disorders (e.g. dementia, amnestic disorders, and age-related cognitive decline (ARCD)), Parkinson's diseases (e.g. dementia in Parkinson's disease, neuroleptic-induced parkinsonism and tardive dyskinesias), endocrine disorders (e.g. hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, attention deficit hyperactivity disorder (ADHD), chronic paroxysmal hemicrania, headache (associated with vascular disorders), emotional lability, pathological crying, sleeping disorder (cataplexy) and shock.

Disorders of particular interest include depression, attention deficit hyperactivity disorder, obsessive-compulsive disorder, post-traumatic stress disorder, substance abuse disorders and sexual dysfunction including (in particular) premature ejaculation. Premature ejaculation may be defined as persistent or recurrent ejaculation before, upon or shortly after penile penetration of a sexual partner. It may also be defined as ejaculation occurring before the individual wishes [see ‘The Merck Manual’, 16^{th} edition, p 1576, published by Merck Research Laboratories, 1992].

Thus, according to further aspects, the invention provides:
i) a compound of the invention for use as a pharmaceutical;
ii) the use of a compound of the invention in the manufacture of a medicament for the treatment or prevention of a disorder in which the regulation of monoamine transporter function is implicated, for example depression, attention deficit hyperactivity disorder, obsessive-compulsive disorder, post-traumatic stress disorder, substance abuse disorders or sexual dysfunction including premature ejaculation;
iii) the use of a compound of the invention in the manufacture of a medicament for the treatment or prevention of premature ejaculation;
iv) a method of treatment or prevention of depression, attention deficit hyperactivity disorder, obsessive-compulsive disorder, post-traumatic stress disorder, substance abuse disorders or sexual dysfunction including premature ejaculation, which comprises administering a therapeutically effective amount of a compound of the invention to a patient in need of such treatment or prevention;
v) a method of increasing ejaculatory latency which comprises the administration of an effective amount of a compound of the invention to a male desiring increased ejaculatory latency; and
vi) a compound of the invention for the treatment or prevention of a disorder in which the regulation of monoamine transporter function is implicated, for example depression, attention deficit hyperactivity disorder, obsessive-compulsive disorder, post-traumatic stress disorder, substance abuse disorders or sexual dysfunction including premature ejaculation.
vii) a compound of the invention for treating premature ejaculation.
viii) a combination including a compound of the invention, and another pharmacologically active agent.

It is to be appreciated that all references herein to treatment include curative, palliative and prophylactic treatment.

Biological Activity

A number of compounds were tested for biological activity by their ability to inhibit the uptake of serotonin by human serotonin transporters as follows.

(i) Cell Culture

Human embryonic kidney cells (HEK-293) stably transfected with either the human serotonin transporter (hSERT), noradrenaline transporter (hNET) or dopamine transporter (hDAT) were cultured under standard cell culture techniques (cells were grown at 37° C. and 5% CO₂ in DMEM-culture media (supplemented with 10% dialysed foetal calf serum (FCS), 2 mM 1-glutamine and 250 μg/ml geneticin)). Cells were harvested for the assay to yield a cell suspension of 750,000 cells/ml.

(i) Determination of Inhibitor Potency

All test compounds were dissolved in 100% DMSO and diluted down in assay buffer to give appropriate test concentrations. Assays were carried out in 96-well filter bottom plates. Cells (7500 cells/assay well) were pre-incubated in standard assay buffer containing either test compound, standard inhibitor or compound vehicle (1% DMSO) for 5 minutes. Reactions were started by addition of either ³H-Serotonin, ³H-Noradrenaline or ³H-Dopamine substrates. All reactions were carried out at room temperature in a shaking incubator. Incubation times were 5 minutes for the hSERT and hDAT assays and 15 minutes for the hNET assay. Reactions were terminated by removal of the reaction mixture using a vacuum manifold followed by rapid washing with ice cold assay buffer. The quantity of ³H-substrate incorporated into the cells was then quantified.

Assay plates were dried in a microwave oven, scintillation fluid added, and radioactivity measured. Potency of test compounds was quantified as IC₅₀ values (concentration of test compound required to inhibit the specific uptake of radiolabelled substrate into the cells by 50%).

(iii) Standard Assay Buffer Composition:

Trizma hydrochloride (26 mM)

NaCl (124 mM)

KCl (4.5 mM)

KH₂PO₄ (1.2 mM)

MgCl₂.6H₂O (1.3 mM)

Ascorbic acid (1.136 mM)

Glucose (5.55 mM)

pH 7.40

CaCl₂ (2.8 mM)

Pargyline (100 μM)

Note: The pH of the buffer was adjusted to 7.40 with 1M NaOH before addition of CaCl₂ and pargyline.

(iv) Summary of Assay Parameters

| | hSERT | hDAT | hNET |
|---|---|---|---|
| | Assay | Assay | Assay |
| Cell concentration per | 75,000 | 75,000 | 75,000 |
| assay well. | | | |
| Substrate Concentration. | ³H-5HT | ³H-Dopamine | ³H-Noradrenaline |
| | (50 nM) | (200 nM) | (200 nM) |
| Incubation time (minutes) | 5 | 5 | 15 |

The compounds of the present invention are a class of selective serotonin reuptake inhibitors, selective over dopamine reuptake.

The compounds of the present invention have a serotonin re-uptake inhibition (SRI) IC₅₀ value of less than or equal to 100 nM. Preferred compounds have a serotonin re-uptake inhibition (SRI) IC₅₀ value of less than or equal to 50 nM. Particularly preferred compounds have a serotonin re-uptake inhibition (SRI) IC₅₀ value of less than or equal to 10 nM.

The compounds of the present invention are more than 10-fold as potent in the inhibition of serotonin re-uptake than in the inhibition of dopamine re-uptake, preferred compounds are more than 100-fold as potent. In a preferred embodiment of the present invention, are compounds which are more than 10-fold as potent in the inhibition of serotonin re-uptake than in the inhibition of noradrenaline re-uptake, particularly preferred compounds are more than 100-fold as potent.

In particular,(N-Isobutyl-N-[(3R)-pyrrolidin-3-yl]-2-naphthamide (Example 23) has a serotonin re-uptake inhibition (SRI) IC₅₀ of 4.7 nM; is 261 fold more potent over noradrenaline re-uptake (has a noradrenaline re-uptake inhibition IC₅₀ of 1230 nM); and is 2018 fold more potent over dopamine re-uptake (has a dopamine re-uptake inhibition IC₅₀ of 9487 nM).

The compounds of the invention may be administered alone or as part of a combination therapy. If a combination of active agents are administered, then they may be administered simultaneously, separately or sequentially. In particular, the compounds of the invention may be combined with the following preferably for the treatment of PE:

i) Alpha-blockers (e.g. phentolamine, doxazasim, tamsulosin, terazasin, prazasin and Example 19 of W09830560. A possible rationale for alpha-blockers treating premature ejaculation is as follows. Muscular activity of the ejaculatory smooth muscles (vas deferens, seminal vesicles and urethra) are controlled by the sympathetic nervous system through the release of noradrenalin. Noradrenalin acts on the alpha 1 adrenoreceptors, stimulating muscle contractions, leading to seminal emission and subsequently ejaculation. Blocking these receptors will therefore inhibit ejaculation.

ii) Apomorphine—teachings on the use of apomorphine as a pharmaceutical may be found in U.S. Pat. No. 5,945,117.

iii) Dopamine D2 agonists (e.g. Premiprixal, Pharmacia Upjohn compound number PNU95666).

iv) Melanocortin receptor agonists (e.g. Melanotan II).

v) PGE1 receptor agonists (e.g. alprostadil).

vi) Mono amine transport inhibitors, particularly Noradrenaline Re-uptake Inhibitors (NRIs) (e.g. Reboxetine), other Serotonin Re-uptake Inhibitors (SRIs) (e.g. paroxetine) or Dopamine Re-uptake Inhibitors (DRIs).

vii) 5-HT3 antagonists (e.g. ondansetron and granisetron). A possible rationale for 5-HT3 antagonists treating premature ejaculation is as follows. 5-HT3 receptors, present in the lumen of the posterior portion of the urethra, are stimulated by 5-HT in the semen during seminal emission, leading to a sensitisation of the spinal relex pathway which leads to ejaculation. Therefore, an antagonist would prevent this sensitisation and thus delay ejaculation.

viii) PDE inhibitors such as PDE2 (e.g. erythro-9-(2-hydroxyl-3-nonyl)-adenine) and Example 100 of EP 0771799-incorporated herein by reference) and in particular a PDE5 inhibitor (e.g. sildenafil, 1-{[3-(3,4-dihydro-5-methyl-4-oxo-7-propylimidazo[5,1-f]-as-trazin-2-yl)-4-ethoxyphenyl]sulfonyl}-4-ethylpiperazine i.e. vardenafil/Bayer BA 38-9456 or IC351 (see structure below, Icos Lilly)). A possible rationale for PDE inhibitors treating premature ejaculation is as follows. cAMP and CGMP levels in the ejaculatory smooth muscles regulate muscle tone of these ejaculatory muscles and so delay ejaculation.

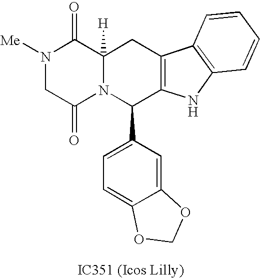

ix) Potassium channel openers.

x) P2X purinergic receptor antagonists.

xi) Endothelin receptor antagonists

xii) 5HT1a antagonists eg robalzoton.

For human use the compounds of the invention can be administered alone but in human therapy will generally be administered in admixture with a suitable pharmaceutical excipient, diluent or carrier selected with regard to the intended route of administration and standard pharmaceutical practice. For example, the compounds of the invention, can be administered orally, buccally or sublingually in the form of tablets, capsules (including soft gel capsules), ovules, elixirs, solutions or suspensions, which may contain flavouring or colouring agents, for immediate-, delayed-, modified-, sustained-, dual-, controlled-release or pulsatile delivery applications. The compounds of the invention may also be administered via intracavernosal injection. The compounds of the invention may also be administered via fast dispersing or fast dissolving dosage forms.

Such tablets may contain excipients such as microcrystalline cellulose, lactose, sodium citrate, calcium carbonate, dibasic calcium phosphate, glycine, and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention, and their pharmaceutically acceptable salts, may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form i.e. within the matrix, and/or on the dosage form, i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The compounds of the invention can also be administered parenterally, for example, intravenously, intra-arterially, intraperitoneally, intrathecally, intraventricularly, intraurethrally, intrasternally, intracranially, intramuscularly or subcutaneously, or they may be administered by infusion techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

The following dosage levels and other dosage levels herein are for the average human subject having a weight range of about 65 to 70 kg. The skilled person will readily be able to determine the dosage levels required for a subject whose weight falls outside this range, such as children and the elderly.

For oral and parenteral administration to human patients, the daily dosage level of the compounds of the invention or salts or solvates thereof will usually be from 10 to 500 mg (in single or divided doses). Thus, for example, tablets or capsules of the compounds of the invention may contain from 5 mg to 250 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will also appreciate that, in the treatment of certain conditions (including PE), compounds of the invention may be taken as a single dose on an “as required” basis (i.e. as needed or desired).

### Example Tablet Formulation

In general a tablet formulation could typically contain between about 0.01 mg and 500 mg of a compound of the invention whilst tablet fill weights may range from 50 mg to 1000 mg. An example formulation for a 10 mg tablet is illustrated:

| Ingredient | % w/w |
|---|---|
| Compound of the invention | 10.000* |
| Lactose | 64.125 |
| Starch | 21.375 |
| Croscarmellose Sodium | 3.000 |
| Magnesium Stearate | 1.500 |

*This quantity is typically adjusted in accordance with drug activity.

The compounds of the invention can also be administered intranasally or by inhalation and are conveniently delivered in the form of a dry powder inhaler or an aerosol spray presentation from a pressurised container, pump, spray or nebulizer with the use of a suitable propellant, e.g. dichlorodifluoromethane, trich lorofluoromethane, dich lorotetra-fluoro-ethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray or nebulizer may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains from 1 to 50 mg of a compound of the invention for delivery to the patient. The overall daily dose with an aerosol will be in the range of from 1 to 50 mg which may be administered in a single dose or, more usually, in divided doses throughout the day.

The compounds of the invention may also be formulated for delivery via an atomiser. Formulations for atomiser devices may contain the following ingredients as solubilisers, emulsifiers or suspending agents: water, ethanol, glycerol, propylene glycol, low molecular weight polyethylene glycols, sodium chloride, fluorocarbons, polyethylene glycol ethers, sorbitan trioleate, oleic acid.

Alternatively, the compounds of the invention can be administered in the form of a suppository or pessary, or they may be applied topically in the form of a gel, hydrogel, lotion, solution, cream, ointment or dusting powder. The compounds of the invention may also be dermally or transdermally administered, for example, by the use of a skin patch. They may also be administered by the ocular, pulmonary or rectal routes.

For ophthalmic use, the compounds can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters, wax, cetearyl alcohol, 2-octyldodecanol, benzyl alcohol and water.

The compounds of the invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/1 1172, WO-A-94/02518 and WO-A-98/55148.

For oral or parenteral administration to human patients the daily dosage levels of compounds of the invention will be from 0.01 to 30 mg/kg (in single or divided doses) and preferably will be in the range 0.01 to 5 mg/kg. Thus tablets will contain 1 mg to 0.4 g of compound for administration singly or two or more at a time, as appropriate. The physician will in any event determine the actual dosage which will be most suitable for any particular patient and it will vary with the age, weight and response of the particular patient. The above dosages are, of course only exemplary of the average case and there may be instances where higher or lower doses are merited, and such are within the scope of the invention.

Oral administration is preferred. Preferably, administration takes place shortly before an effect is required.

For veterinary use, a compound of the invention is administered as a suitably acceptable formulation in accordance with normal veterinary practice and the veterinary surgeon will determine the dosing regimen and route of administration which will be most appropriate for a particular animal.

Thus according to a further aspect, the invention provides a pharmaceutical formulation containing a compound of the invention and a pharmaceutically acceptable adjuvant, diluent or carrier.

The invention is illustrated by the following non-limiting examples in which the following abbreviations and definitions are used:
Arbocel® filter agent
br broad
Boc tert-butoxycarbonyl
CDI carbonyidiimidazole
δ chemical shift
d doublet
Δ heat
DCCI dicyclohexylcarbodiimide
DCM dichloromethane
DMF N,N-dimethylformamide
DMSO dimethylsulfoxide
ES⁺ electrospray ionisation positive scan
ES⁻ electrospray ionisation negative scan
Ex Example
h hours
HOBt 1-hydroxybenzotriazole
HPLC high pressure liquid chromatography
m/z mass spectrum peak
min minutes
MS mass spectrum
NMR nuclear magnetic resonance
Prec precursor
Prep preparation
q quartet
s singlet
t triplet
Tf trifluoromethanesulfonyl
TFA trifluoroacetic acid
THF tetrahydrofuran
TLC thin layer chromatography
TS⁺ thermospray ionisation positive scan
WSCDI 1-(3-dimethylaminopropyl)-3-ethylcarbodiimide hydrochloride

¹H Nuclear magnetic resonance (NMR) spectra were in all cases consistent with the proposed structures. Characteristic chemical shifts (δ) are given in parts-per-million downfield from tetramethylsilane using conventional abbreviations for designation of major peaks: e.g. s, singlet; d, doublet; t, triplet; q, quartet; m, multiplet; br, broad. The following abbreviations have been used for common solvents: CDCl₃, deuterochloroform; DMSO, dimethylsulfoxide. The abbreviation psi means pounds per square inch and LRMS means low resolution mass spectrometry. Where thin layer chromatography (TLC) has been used it refers to silica gel TLC using silica gel 60 F₂₅₄ plates, R_{f} is the distance travelled by a compound divided by the distance travelled by the solvent front on a TLC plate. Melting points were determined using a Perkin Elmer DSC7 at a heating rate of 20° C./minute).

Where indicated, compounds were characterised as their hydrochloride salts. The procedure can be carried out with other solvents e.g. diethyl ether or DCM.

Commercial starting materials were obtained from Aldrich Chemical Co, Lancaster Synthesis Ltd or Acros Organics.

### EXAMPLES 1 to 15

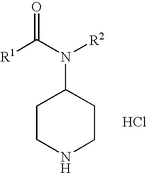

A solution of HCl in dioxan (5-25 eq, 4M solution) was added to a solution of the protected amines (1 eq) in dichloromethane (approx 5-10 mlmmol⁻¹), and the solution was stirred at room temperature for between 24 and 72 hours. The reaction was concentrated under reduced pressure and the residue was azeotroped with dichloromethane and then triturated with pentane. The supernatant was removed and the product was dried in vacuo to afford the title compounds.

| Ex | R¹ | R² | precursor | Form | Data |
|---|---|---|---|---|---|
| 1¹ | 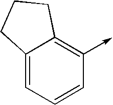 | 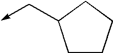 | Prep 50 | oil | ¹H-nmr (CD₃OD, 400 MHz) δ 1.01 (br, 1H), 1.40 (br, 3H), 1.63 (br, 3H), 2.05 (m, 4H), 2.80 (br, 4H), 2.96 (t, 3H), 3.02-3.23 (m, 3H), 3.50 (br, 3H), 3.81 (br, 2H), 7.04 (d, 1H), 7.10 (t, 1H), 7.28 (d, 1H) LRMS: m/z APCI⁺ 327 [MH⁺] |
| 2 | 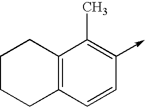 | 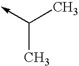 | Prep 51 | White solid | ¹H-nmr (DMSO-d₆, 400 MHz) δ 1.05 (m, 6H), 1.60-1.67 (d, 4H), 2.00 (s, 3H), 2.58 (s, 2H), 2.70 (s, 2H), 2.95 (d, 4H), 3.3 (t, 4H), 3.45 (d, 1H), 3.55 (t, 1H), 6.80 (d, 1H), 6.90 (d, 1H), 8.35 (s, 1H), 9.21 (s, 1H). LRMS: m/z APCI⁺ 315 [MH⁺] |
| 3 | 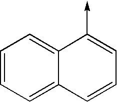 | 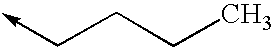 | Prep 41 | White solid | ¹H-nmr (CD₃OD, 400 MHz) δ rotamers: 0.55, 1.08 (2xt, 3H), 0.89-0.97 (m, 2H), 1.30-4.30 (m, 13H), 7.41-7.46 (m, 1H), 7.53-7.60 (m, 3H), 7.71-7.78 (m, 1H), 7.94-7.99 (m, 2H). LRMS: m/z APCI⁺ 311 [MH⁺]. |
| 4 | 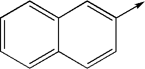 | 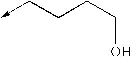 | Prep 77 | Yellow foam | ¹H-nmr (CD₃OD, 400 MHz) δ: 1.20-2.20 (brm, 10H), 3.00-4.20 (brm, 6H), 7.46 (1H, d), 7.57 (m, 2H), 7.89-7.96 (m, 2H), 7.99 (1H, d) LRMS: m/z APCI⁺ 327 [MH⁺]Microanalysis found: C, 61.07; H, 7.59; N, 6.74. C₂₀H₂₆N₂O₂; HCl: 1.7H₂O requires C, 61.04; H, 7.79; N, 7.12%. |
| 5² | 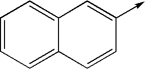 | 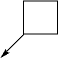 | Prep 43 | White solid | ¹H-nmr (CD₃OD, 400 MHz) δ: 1.42-1.49 (m, 1H), 1.67-1.75 (m, 1H), 1.96-1.99 (m, 2H), 2.04-2.11 (m, 2H), 2.42 (br, 2H), 3.01 (br, 2H), 3.19 (m, 2H), 3.49-3.52 (m, 2H), 3.86-3.94 (m, 1H), 4.27 (q, 1H), 7.43 (dd, 1H), 7.55-7.58 (m, 2H), 7.87 (s, 1H). 7.90-7.97 (m, 3H) LRMS: m/z APCI⁺ 309 [MH⁺]. |
| 6 | 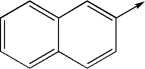 | 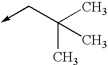 | Prep 42 | | ¹H-nmr (DMSO-d₆, 400 MHz) δ: 0.95 (br, 9H), 1.90 (br, 4H), 2.75 (br, 2H), 3.30 (br, 4H), 3.85 (br, 1H), 7.50 (br, 3H), 7.95 (br, 3H), 8.65 (br, 1H) LRMS: m/z APCI⁺ 325 [MH⁺]Microanalsyis found: C, 66.57; H, 8.26; N, 7.45. C₂₁H₂₈N₂O; HCl; H₂O requires C, 66.56; H, 8.25; N, 7.39%. |
| 7³ | 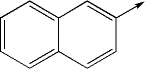 | 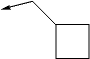 | Prep 45 | | ¹H-nmr (CD₃OD, 400 MHz) δ: Rotamers: 1.5-1.7 (br, 2H), 2.1 (m, 4H), 2.6-3.5 (br, 6H), 3.3.4-3.6 (br, 4H), 3.8 (m, 2H), 7.4 (d, 1H), 7.6 (m, 2H), 7.8-8.0 (m, 4H) LRMS: m/z ES⁺ 323 [MH⁺] |
| 8⁴ | 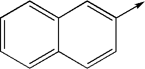 | 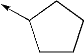 | Prep 44 | White solid | ¹H-nmr (CD₃OD, 400 MHz) δ: rotamers: 1.47 (br, 2H), 1.82 (br, 6H), 1.98-2.01 (br, m, 2H), 3.10 (br, 4H), 3.47-3.57 (br, m, 3H), 3.98 (br, 1H), 7.43 (dd, 1H), 7.54-7.58 (m, 2H), 7.86 (s, 1H), 7.90-7.97 (m, 3H). LRMS: m/z APCI⁺ 323 [MH⁺]. |
| 9 | 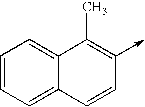 | 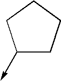 | Prep 75 | White solid | ¹H-nmr (CD₃OD, 400 MHz) δ: rotamers: 1.30-2.80 (m, 13H), 3.15-3.89 (m, 8H), 7.22-7.26 (m, 1H), 7.53-7.62 (m, 2H), 7.82 (d, 1H), 7.89 (d, 1H), 8.10 (d, 1H). LRMS: m/z APCI⁺ 337 [MH⁺]. |
| 10 | 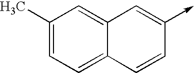 | 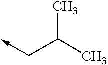 | Prep 56 | | ¹H-nmr (CD₃OD, 400 MHz) δ: 0.60-1.20 (br, 6H), 1.95 (br, 1H), 2.10 (br, 2H), 2.55 (s, 3H), 2.70 (br, 2H), 3.10 (br, 2H), 3.30 (d, 2H), 3.50 (br, 2H), 3.90 (br, 1H), 7.35 (d, 1H), 7.45 (d, 1H), 7.70 (s, 1H), 7.80 (m, 2H), 7.90 (d, 1H) LRMS: m/z APCI⁺ 325 [MH⁺]Microanalysis found: C, 69.14; H, 8.37; N, 7.31. C₂₁H₂₈N₂O; HCl; 0.23 |
| # requires C, 69.09; H, 8.13; N, 7.67% | | | | | |
| 11¹ | 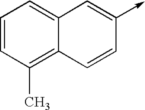 | 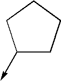 | Prep 55 | Off- white | solid ¹H-nmr (CD₃OD, 400 MHz) δ: 1.35-1.50 (br, 2H), 1.70-1.90 (br, 2H), 1.65-2.05 (2xbr, 4H), 1.65-2.05 (2xbr, 4H), 2.70 (s, 3H), 2.85-3.80 (3xbr, 5H), 3.90-4.05 (br, 1H), 7.40-7.50 (m, 3H), 7.75 (s, 1H), 7.85 (s, 1H), 8.15 (s, 1H) LRMS: m/z APCI⁺ 337 [MH⁺] |
| 12¹ | 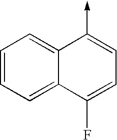 | 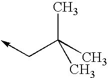 | Prep 53 | White solid | ¹H-nmr (CD₃OD, 400 MHz) δ (rotamers) : 0.75-1.15 (brs, 9H), 2.20-2.40 (2xd, 1H), 3.05 (t, 3H), 3.15-3.35 (m, 4H), 3.50-3.60 (m, 1H), 3.50-3.60 (m, 2H), 7.25 (t, 1H), 7.45-7.55 (2xt, 1H), 7.65 (m, 2H), 7.75 (d, 1H), 8.00-8.10 (2xd, 1H) LRMS: m/z APCI⁺ 343 [MH⁺]Microanalysis found: C, 65. |
| # C₂₁H₂₇FN₂O; HCl; 0.85H₂O requires C, 65.21; H, 8.13; N, 6.88%. | | | | | |
| 13⁵ | 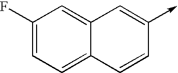 | 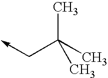 | Prep 54 | | ¹H-nmr (CD₃OD, 400 MHz): Rotamers: 0.88 (s, 9H), 1.18 (br, 2H), 1.9 (br, 2H), 2.3 (2xm, 2H), 3.0-3.4 (br, 3H), 3.55 (br, 2H), 7.25 (t, 1H), 7.40-7.45 (br, 1H), 7.65 (m, 2H), 7.8-8.0 (br, 1H), 8.2 (d, 1H) LRMS: m/z (APCI), [MH⁺] 343 |
| 14 | 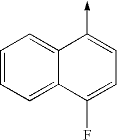 | 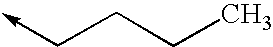 | Prep 52 | White powder | ¹H-nmr (CD₃OD, 400 MHz): Rotamers: 0.59 (t, 3H), 0.85 (m, 2H), 1.1 (t, 2H), 1.35 (m, 3H), 1.8-2.2 (br, 4H), 2.6 (m, 1H), 3.0-3.2 (br, 3H), 7.25 (m, 1H), 7.42 (m, 1H), 7.65 (m, 2H), 7.8 (m, 1H), 8.2 (brm, 1H). LRMS: m/z ES⁺ 329 [MH]⁺ |
| 15^{1,} | 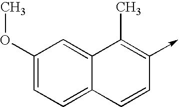 | 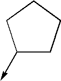 | Prep 57 | | ¹H-nmr (CD₃OD, 400 MHz): Rotamers: 1.4 (m, 2H), 1.7 (m, 6H), 2.0 (m, 6H), 2.6 (s, 3H), 3.1 (m, 2H), 3.5 (m, 2H), 4.0 (s, 3H), 7.1 (d, 1H), 7.2 (d, 1H), 7.7 (s, 1H), 7.7 (d, 1H), 7.8 (d, 1H) LRMS: m/z ES⁺ 367 [MH⁺] |

¹= the residue was triturated with ether
²= product crystallised out on addition of pentane
³= dioxan was the reaction solvent
⁴= HCl in ether (2M) was used
⁵= no dichloromethane was used in the reaction

### EXAMPLE 16

### N-Butyl-3-chloro-2-methyl-N-piperidin-4-ylbenzamide

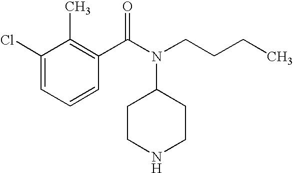

A solution of the compound from preparation 40 (243 mg, 0.60 mmol) in dichloromethane (10 ml) and 2M HCl in ether (10mi) was stirred at room temperature for 18 hours. The solution was concentrated under reduced pressure and the residue was purified by column chromatography on silica gel using an elution gradient of dichloromethane:methanol:0.88 ammonia (99:1:0.1 to 85:15:1.5) to give the title compound as a colourless oil, 169 mg.

¹H-nmr (CDCl₃, 400MHz) δ: 0.80 (m, 2H), 0.95 (m, 3H), 1.30-2.10 (m, 9H), 2.20-2.40 (m, 5H), 2.80-3.40 (m, 7H), 5.10 (m, 2H), 7.00 (m, 1H), 7.10 (m, 1H), 7.30 (m, 1H).

LRMS: m/z APCI⁺ 309, 311 [MH⁺],

### EXAMPLE 17

### 3-Chloro-N-(cyclopropylmethyl)-2-methyl-N-piperidin-4-ylbenzamide

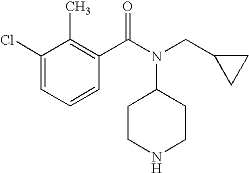

A solution of the protected amine from preparation 46 (333 mg, 0.82 mmol) and trifluoroacetic acid (0.5 ml) in dichloromethane (5 ml) was stirred at room temperature for 24 hours. The reaction was concentrated under reduced pressure and the residue was azeotroped with toluene, then dissolved in methanol. The solution was treated with 0.88 ammonia, and pre-adsorbed onto silica gel. This was purified by column chromatography on silica gel using an elution gradient of dichloromethane:methanol:0.88 ammonia (98:2:0.2 to 94:6:0.6) to afford the title compound as an oil, 140 mg.

¹H-nmr (CD₃OD, 400 MHz) δ: (rotamers): −0.09, 0.49, 0.86 and 1.21 (4×m, 4H), 1.77 (m, 4H), 2.19 (m, 3H), 2.31 and 2.34 (2×s, 3H), 2.71 (m, 1H), 3.00 (m, 2H), 3.16 (m, 1H), 3.29 (m, 1H), 3.43 and 4.26 (2×m, 1H), 7.13 (m, 1H), 7.26 (m, 1H), 7.44 (m, 1H).

LRMS: m/z APCI⁺ 307/309 [MH⁺]

### EXAMPLE 18

### 3-Chloro-N-ethyl-2-methyl-N-Diieridin-4-ylbenzamide hydrochloride

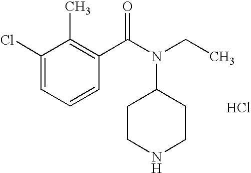

A 2M solution of HCl in MeOH was added to the protected amine from preparation 48 (69 mg, 0.18 mmol) and the resulting solution was stirred at room temperature for 18 hours. The mixture was evaporated under reduced pressure and the residue was purified by column chromatography on silica gel using dichloromethane:methanol:0.88 ammonia (96:4:0.4 to 88:12:1.2) as eluant. The product was then treated with 2M HCl in ether, and the solution evaporated under reduced pressure to afford the title compound, 43 mg.

¹H-nmr (CDCl₃, 400 MHz) δ rotamers : 0.95, 1.25 (2×t, 3H), 1.50-1.85 (m, 4H), 2.25 (2×s, 3H), 2.70-3.60 (m, 4H), 4.00-4.30 (m, 2H), 7.00-7.40 (m, 3H).

LRMS: m/z APCI⁺ 281, 283 [MH⁺]

### EXAMPLE 19

### 3-Chloro-2-methyl-N-piperidin-4-yl-N-(4,4,4-trifluorobutyl)benzamide hydrochloride

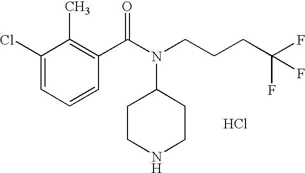

A solution of the protected amine from preparation 47 (375 mg, 0.81 mmol) and trifluoroacetic acid (3 ml) in dichloromethane (3 ml) was stirred at room temperature for 24 hours. The reaction was concentrated under reduced pressure and the residue was azeotroped with toluene, then dissolved in methanol. The solution was treated with 0.88 ammonia, and pre-adsorbed onto silica gel. This was purified by column chromatography on silica gel using an elution gradient of dichloromethane:methanol:0.88 ammonia (98:2:0.2 to 94:6:0.6). The product was treated with a 2M solution of HCl in ether. This solution was then concentrated and azeotroped with toluene to afford the title compound as a white foam, 150 mg.

¹H-nmr (DMSO-d₆, 400 MHz) δ (rotamers): 1.58 (m, 1H), 1.74 (m, 2H), 1.98 (m, 4H), 2.19 and 2.21 (2×s, 3H), 2.30 (m, 2H), 2.62 and 2.81 (2×m, 1H), 2.99 (m, 1H), 3.19 (m, 1H), 3.39 (m, 3H), 4.20 (m, 1H), 7.19 (m, 1H), 7.27 (m, 1H), 7.46 (m, 1H).

LRMS: m/z APCI⁺ 363/365 [MH⁺]

### EXAMPLE 20

### 3-Chloro-2-methyl-N-pentyl-N-piperidin-4-ylbenzamide

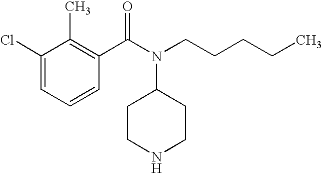

A mixture of 3-chloro-2-methyl-benzoic acid (200 mg, 1.12 mmol), the amine from preparation 33 (349 mg, 1.29 mmol), 1-hydroxybenzotriazole hydrate (243 mg, 1.8 mmol), 1-(3-dimethylaminopropyl)-3-ethylcarbodiimide hydrochloride (345 mg, 1.80 mmol) and triethylamine (360 μl, 2.6 mmol) in dichloromethane (10 ml) was stirred at room temperature for 18 hours. The mixture was washed with saturated aqueous sodium bicarbonate solution, and evaporated under reduced pressure. The residue was purified by column chromatography on silica gel using an elution gradient of pentane:dichloromethane:methanol (50:50:0 to 0:100:0 to 0:95:5). The product was dissolved in dichloromethane and treated with a 2M solution of HCl in ether. This solution was then evaporated under reduced pressure to afford the title compound as a white solid, 173 mg.

¹H-nmr (CDCl₃, 400 MHz) δ: 0.75-1.10 (m, 5H), 1.35 (m, 3H),1.50-1.95 (m, 5H), 2.00 (s,1H), 2.20-2.40 (m, 4H), 2.70-3.50 (m, 6H), 6.95-7.40 (m, 3H).

LRMS: m/z APCI⁺ 323/325 [MH⁺]

### EXAMPLE 21

### N-Butyl-N-[(3S)-pyrrolidin-3-yl]-5,6,7,8-tetrahydronaphthalene-2-carboxamide

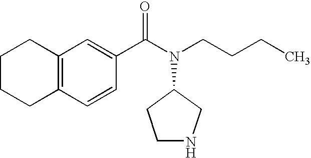

A mixture of the protected amine from preparation 63 (0.8 mmol), potassium carbonate (0.22 g, 1.6 mmol), methanol (5 ml) and water (0.5 ml) was stirred at room temperature for 18 hours. The reaction mixture was pre-adsorbed onto silica gel and purified by column chromatography on silica gel using ethyl acetate:methanol:0.88 ammonia (90:10:1), followed by dichloromethane:methanol:0.88 ammonia (90:10:1) as eluants to give the title compound as an oil, 96 mg.

¹H-nmr (CD₃OD, 400 MHz) δ: 0.82 (m, 3H), 1.20 (m, 2H), 1.59 (m, 2H), 1.81 (m, 4H), 2.78 (m, 4H), 3.07 (m, 1H), 3.27 (m, 6H), 3.58 (m, 1H), 4.27 (m, 1H), 7.10 (m, 3H).

LRMS: m/z APCI⁺ 301 [MH⁺]

### EXAMPLE 22

### N-Isobutyl-1-methyl-N-[(3S)-pyrrolidin-3-yl]-5,6,7,8-tetrahydronaphthalene-2-carboxamide

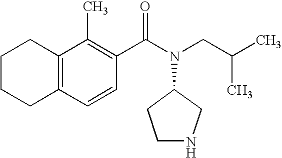

The title compound was obtained as an oil in 65% yield from the protected amine from preparation 66 following the procedure described in example 21.

¹H-nmr (CD₃OD, 400 MHz) δ: 0.82 (m, 6H), 1.80 (m, 4H), 2.10 (m, 3H), 2.79 (m, 4H), 3.02 (m, 1H), 3.25 (m, 6H), 7.09 (m, 3H).

LRMS m/z APCI⁺ 301 [MH⁺]

### EXAMPLE 23

### N-Isobutyl-N-[(3R)-pyrrolidin-3-yl]-2-naphthamide hydrochloride

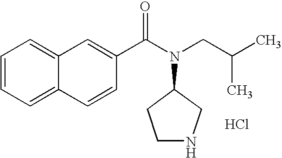

Potassium carbonate (98 mg, 0.71 mmol) and water (0.5 ml) were added to a solution of the protected amine from preparation 73 (140 mg, 0.36 mmol) in methanol (5 ml) and the solution was stirred at room temperature for 18 hours. The mixture was partitioned between ethyl acetate (50 ml) and 10% aqueous potassium carbonate solution, the layers were separated, and the organic phase was dried (MgSO₄) and evaporated under reduced pressure. The residue was dissolved in ethyl acetate, a solution of HCl in ether (2M, 2 ml) was added and the solution was stirred at room temperature for 18 hours. The mixture was evaporated under reduced pressure to give the title compound, 34 mg.

¹H-nmr (CD₃OD, 400 MHz) δ: 0.79 (br, 6H), 1.86-2.07 (m, 4H), 2.57 (br, 2H), 3.57 (brt, 1H), 3.82 (br, 2H), 4.37 (br, 1H) 7.49 (d, 1H), 7.60 (t, 2H), 7.83-8.04 (m, 4H)

LRMS: m/z APCI⁺ 297 [MH⁺]

### EXAMPLE 24

### N-Isobutyl-N-[(3R)-pyrrolidin-3-yl]quinoline-6-carboxamide hydrochloride

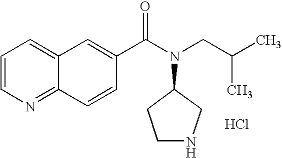

The title compound was obtained from the protected amine from preparation 69 following the procedure described in example 23.

¹H-nmr (CD₃OD, 400 MHz) δ: 0.79 (brs, 6H), 1.96 (br, 1H), 2.59 (br, 2H), 3.20-3.32 (m, 3H), 3.60 (brt, 1H), 3.83 (br, 2H), 4.41 (br, 1H), 8.15-8.24 (m, 2H), 8.36 (d, 1H), 8.48 (s, 1H), 9.25-9.37 (m, 2H)

LRMS: m/z APCI 298 [MH⁺]

### EXAMPLE 25

### N-Isobutyl-N-[(3S)-pyrrolidin-3-yl]quinoline-6-carboxamide hydrochloride

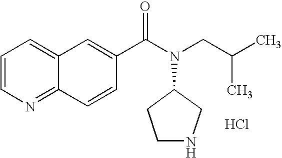

The title compound was obtained from the protected amine from preparation 68 following the procedure described in example 23.

¹H-nmr (CD₃OD, 400 MHz) δ: 0.77 (br, 6H), 1.96 (br, 1H), 2.60 (br, 1H), 3.28 (m, 3H), 3.61 (t, 1H), 3.84 (s, 2H), 4.41 (br, 1H), 8.20 (m, 2H), 8.39 (d, 1H), 8.46 (s,1H), 9.29 (m, 2H)

LRMS: m/z APCI⁺ 298 [MH⁺]

### EXAMPLE 26

### N-(3-Methylbutyl)-N-[(3R)-pyrrolidin-3-yl]-2-naphthamide hydrochloride

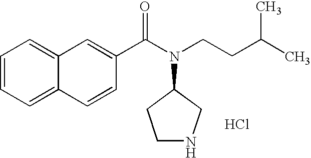

A mixture of the protected amine from preparation 72 (220 mg, 0.54 mmol), and potassium carbonate (149 mg, 1.08 mmol) in methanol (5 ml) and water (0.5 ml) was stirred at room temperature for 18 hours. The mixture was concentrated under reduced pressure and the residue was purified by column chromatography on silica gel using dichloromethane:methanol (100:0 to 94:6). The product was dissolved in dichloromethane, 2M ethereal HCl was added, and this solution was concentrated under reduced pressure. The residue was triturated with ether (3×) and the resulting solid was dried in vacuo to give the title compound, 60 mg.

¹H-nmr (DMSO-d₆, 400 MHz) δ: 1.6-1.8 (br, 6H), 1.3-1.5 (br, 2H), 2.1-2.3 (br, 2H), 3.1-3.4 (br, 4H), 3.7 (br, 2H), 4.4 (m, 1H), 7.5 (d, 1H), 7.6 (m, 2H), 7.9-8.0 (m, 4H), 9.6 (br, 1H)

LRMS m/z APCI⁺ 311 [MH⁺]

Microanalysis found: C, 65.24; H, 7.92; N, 7.60. C₂₀H₂₆N₂O; HCl:1.10H₂O requires C, 65.49; H, 8.03; N, 7.64%.

### EXAMPLE 27

### N-Butyl-1-methyl-N-[(3R)-pyrrolidin-3-yl]-5,6,7,8-tetrahydronaphthalene-2-carboxamide hydrochloride

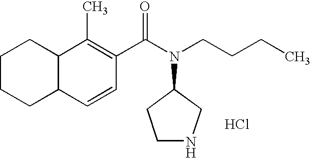

The title compound was obtained from the protected amine from preparation 65, following the procedure described in example 26.

¹H-nmr (DMSO-d₆ 400MHz) δ 0.63 (t, 3H), 0.9-1.05 (2m, 2H), 1.25 (m, 2H), 1.6-1.8 (2t, 4H), 2.00 (s, 2H), 2.45 (s, 2H), 2.65 (t, 2H), 3.00-3.60 (5m, 7H), 6.80-7.00 (2d, 2H).

LRMS m/z APCI⁺ 315 [MH⁺]

### EXAMPLE 28

### Methyl 2-({2-naphthoyl[(3R)-pyrrolidin-3-yl]amino}methyl)benzoate hydrochloride

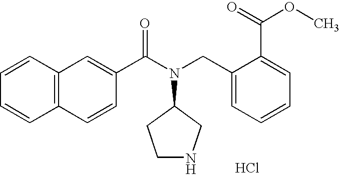

The title compound was obtained from the protected amine from preparation 74, following the procedure described in example 26.

¹H-nmr (DMSO-d₆, 400 MHz) δ: 2.2 (m, 2H), 3.2-3.4 (m, 4H), 3.8 (s, 3H), 4.5-4.6 (m, 2H), 4.8-4.9 (m, 1H), 7.5-7.7 (m, 7H), 7.8 (m, 2H), 7.9-8.0 (m, 2H), 9.4-9.6 (br, 1H)

LRMS m/z APCI⁺ 389 [MH⁺]

### EXAMPLE 29

### 3,4-Dichloro-N-isopropyl-2-methyl-N-[(3R)-pyrrolidin-3-yl]benzamide hydrochloride

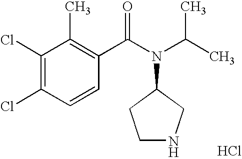

The title compound was obtained from the protected amine from preparation 59, following the procedure described in example 26.

¹H-nmr (CDCl₃, 400 MHz) δ: 1.00-1.20 (m, 6H), 1.90-2.40 (m, 5H), 2.50-2.80 (br, 3H), 2.90-3.80 (m, 5H), 6.90 (d, 1H), 7.35 (d, 1H).

LRMS: m/z APCI⁺ 317 [MH⁺]

### EXAMPLE 30

### N-Butyl-N-[(3R)-pyrrolidin-3-yl]-2-naphthamide oxalate

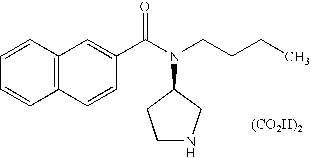

Potassium carbonate (241 mg, 1.74 mmol) was added to a solution of the protected amine from preparation 70 (342 mg, 0.87 mmol) in methanol (8 ml) and water (1 ml), and the reaction mixture was stirred at room temperature for 18 hours. The mixture was partitioned between ethyl acetate (50 ml) and aqueous sodium bicarbonate solution (50 ml), and the layers were separated. The organic phase was dried (MgSO₄) and evaporated under reduced pressure. The residue was dissolved in ethyl acetate, oxalic acid (1 eq) was added and the solution was concentrated under reduced pressure. The residue was triturated with ether and the resulting solid was filtered and dried to afford the title compound as a white crystalline solid, 78 mg.

¹H -nmr (CD₃OD, 400 MHz) δ: 0.75 (br, 3H), 1.15 (br, 2H), 1.60 (br, 2H), 2.40-2.60 (2×br, 2H), 3.20-3.60 (2×t, 2H), 3.35 (t, 2H), 3.70-3.85 (br, 2H), 4.30-4.40 (br, 1H), 7.50 (d, 1H), 7.60 (m, 2H), 7.80-8.00 (m, 4H)

LRMS: m/z APCI⁺ 297 [MH⁺]

### EXAMPLE 31

### N-Benzyl-N-[(3R)-pyrrolidin-3-yl]-2-naphthamide oxalate

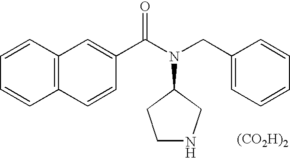

The title compound was obtained as a white crystalline solid from the protected amine from preparation 71 following the procedure described in example 30.

¹H -nmr (CD₃OD, 400 MHz) δ: 2.25-2.50 (2×br, 2H), 3.15 (dd, 1H), 3.35 (t, 1H), 3.60-3.80 (br, 2H), 4.30-4.45 (br, 1H), 4.60-4.70 (s, 2H), 7.25-7.40 (m, 5H), 7.50-7.65 (m, 3H), 7.85-8.00 (m, 3H), 8.00-8.10 (s, 1H)

LRMS: m/z APCI⁺ 331 [MH⁺]

### EXAMPLE 32

### N-Butyl-1-chloro-N-[(3R)-pyrrolidin-3-yl]-2-naphthamide

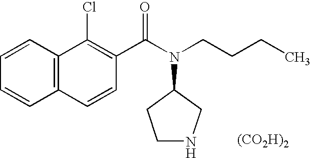

A mixture of the protected pyrrolidine from preparation 78 (270 mg, 0.63 mmol), and potassium carbonate (175 mg, 1.26 mmol) in methanol (5 ml) and water (1 ml) was stirred at room temperature for 2 hours. The reaction mixture was concentrated under reduced pressure, the residue was partitioned between aqueous sodium bicarbonate solution and ethyl acetate and the layers were separated. The organic phase was washed with brine, dried (MgSO₄) and evaporated under reduced pressure. The residue was dissolved in ethyl acetate, oxalic acid (2eq) was added, and the solution was evaporated under reduced pressure. The residue was triturated with ether, the supernatant was decanted off and the product was dried in vacuo to give the title compound, 124 mg.

¹H-nmr (CD₃OD, 400 MHz) δ: 0.70 (t, 3H), 1.10 (m, 2H), 1.55 (m, 2H), 2.55 (m, 2H), 3.25 (m, 3H), 3.60 (m, 1H), 3.85 (m, 2H), 4.45 (m, 1H), 7.45 (m, 1H), 7.70 (m, 2H), 8.00 (m, 2H), 8.35 (d, 1H)

HRMS: m/z (ESI+) 331.1572 [MH⁺]

### EXAMPLE 33

### N-Isobutyl-3-methoxy-2-methyl-N-[(3R)-pyrrolidin-3-yl]benzamide hydrochloride

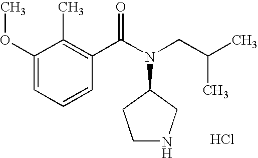

A solution of the protected amine from preparation 60 (470 mg, 1.21 mmol) in methanol (12 ml) and 0.88 ammonia (3 ml) was stirred at room temperature for 18 hours. The mixture was evaporated under reduced pressure and the residue was purified by column chromatography on silica gel using ethyl acetate:methanol:0.88 ammonia (100:0:0 to 75:22.5:2.5). The product was dissolved in ethyl acetate, a 2M solution of HCl in ether was added and the solution was evaporated under reduced pressure to afford the title compound as an oil, 116 mg.

¹H-nmr (CD₃OD, 400 MHz) δ: 0.78 (d, 6H), 1.85 (m, 1H), 2.11 (s, 3H), 2.42-2.62 (m, 2H), 2.98 (m, 1H), 3.09-3.34 (m, 2H), 3.51 (t, 3H), 3.70-3.82 (m, 2H), 3.83 (s, 3H), 4.25 (m, 1H), 6.79 (m, 1H), 7.00 (d, 1H), 7.24 (t, 1H)

LRMS: m/z APCI⁺ 291 [MH⁺]

### EXAMPLE 34

### 3-Chloro-N-isobutyl-4-methyl-N-[(3R)-pyrrolidin-3-yl]benzamide hydrochloride

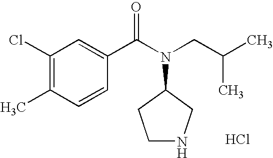

The title compound was obtained from the protected amine from preparation 61 following the procedure described in example 33.

¹H-nmr (CD₃OD, 400 MHz): 0.79 (d, 6H), 1.95 (m, 1H), 2.40 (s, 3H), 2.42-2.59 (m, 2H), 3.20 (d, 3H), 3.37-3.54 (m, 2H), 3.71 (m, 2H), 4.29 (br, 1H), 7.23 (d,1H), 7.39 (d, 1H), 7.00 (d, 1H), 7.42 (s, 1H)

LRMS: m/z APCI⁺ 295 [MH⁺]

### PREPARATION 1

### 2-Formyl-7-methoxy-1-naphthyl trifluoromethanesulfonate

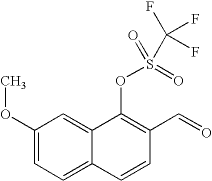

A solution of N-phenyltrifluoromethanesulphonimide (39.8 g, 111 mmol) in dichloromethane (125 ml) was added dropwise to a solution of 1-hydroxy-7-methoxy-naphthalene-2-carbaldehyde (Eur.J.Med. Chem. 1987, 22(4),363-5) (15.0 g, 74.2 mmol), N-diisopropylethylamine (25.9 ml, 148 mmol) and 4-dimethylaminopyridine (452 mg, 3.7 mmol) in dichloromethane (100 ml), and once the addition was complete, the reaction was stirred at room temperature for 1.5 hours. The reaction was quenched by the addition of saturated aqueous sodium bicarbonate solution (10 ml), and then diluted with water (40 ml). The layers were separated, the aqueous was extracted with dichloromethane (3x30 ml), and the combined organic solutions were dried (MgSO₄) and evaporated under reduced pressure. The residual oil was purified by column chromatography on silica gel using an elution gradient of pentane:ethyl acetate (100:0 to 50:50), and the product was recrystallised from dichloromethane:pentane to afford the title compound. The mother liquors were repurified by column chromatography on silica gel using an elution gradient of pentane:dichloromethane (100:0 to 50:50) to provide additional product, 22.5 g in total.

¹H-nmr (CDCl₃, 400 MHz): 4.00 (s, 3H), 7.35 (d, 1H), 7.45 (s, 1H), 8.15 (m, 3H), 10.40 (s, 1H)

### PREPARATION 2

### 7-Methoxy-1-methyl-naphthalene-2-carbaldehyde

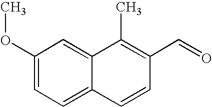

Trimethylboroxine (5.63 g, 44.9 mmol) was added to a mixture of the triflate from preparation 1 (10 g, 29.9 mmol), tetrakis(triphenylphosphine)palladium(0) (1.73 g, 1.5 mmol) and potassium carbonate (12.40 g, 89.7 mmol) in dioxan (150 ml), and the reaction was stirred at 95° C. for 1 hour. Water (1 ml) was added, and the mixture stirred for a further 4 hours at 95° C. The cooled mixture was filtered through Celite® and the filtrate was evaporated under reduced pressure. The residue was purified by column chromatography on silica gel using an elution gradient of pentane:ethyl acetate (100:0 to 50:50) to afford the title compound as a yellow solid, 5.27 g.

¹H-nmr (CDCl₃, 400 MHz): 3.00 (s,3H), 4.00 (s, 3H), 7.30 (d, 1H), 7.40 (s, 1H), 7.70 (d,1H), 7.80 (m, 2H)

LRMS: m/z ES+201 [MH⁺]

### PREPARATION 3

### 7-Methoxy-1-methyl-2-naphthoic acid

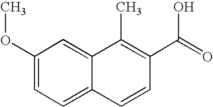

The aldehyde from preparation 2 (5.0 g, 25 mmol), 2-phenyl-1,2-benzisoselenazol-3(2H)-one (1.37 g, 4.1 mmol) and tert-butyl hydroperoxide (70% w/v in water, 6.40 ml) were added to tert-butanol (7.5 ml) and the mixture was heated to 75° C., and stirred for 1.5 hours. 10% Palladium on charcoal (50 mg) was added and the mixture was stirred for a further 2 hours, then concentrated under reduced pressure. The residue was suspended in dichloromethane (25 ml), 5% hydrochloric acid was added, and the biphasic mixture was stirred for 4 hours. This was then filtered, the solid was washed with cold dichloromethane, and the filtrate was evaporated under reduced pressure to afford the title compound, 4.98 g.

¹H-nmr (CD₃OD, 400 MHz): 2.85 (s, 3H), 3.95 (s, 3H), 7.20 (d, 1H), 7.45 (s, 1H), 7.65 (m, 2H), 7.80 (d, 1H).

LRMS: m/z (ES-), [M−H] 215

### PREPARATION 4

### 7-Hydroxy-2-naphthoic acid

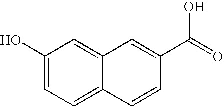

A solution of 2-cyano-7-methoxynaphthalene (Helv. Chim. Acta. 80; 3; 1997; 892-6) (5.88 g, 32 mmol) in 48% hydrobromic acid (200 ml) was heated under reflux for 4 hours, allowed to cool to room temperature and stirred for a further 18 hours. The resulting precipitate was filtered off, washed with water and dried to afford the title compound as a white solid, 5.73 g.

¹H-nmr (DMSO-d₆, 400 MHz) δ: 7.20 (d, 1H), 7.25 (s, 1H), 7.70 (d, 1H), 7.80 (m, 2H), 8.35 (s, 1H), 9.95 (s, 1H), 12.90 (s, 1H)

### PREPARATION 5

### Methyl 1-hydroxy-5,6,7,8-tetrahydronaphthalene-2-carboxylate

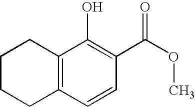

Trimethylsilyl diazomethane (2M in hexane, 17 ml, 34 mmol) was added dropwise to a solution of 1-hydroxy-5,6,7,8-tetrahydronaphthalene-2-carboxylic acid (Justus. Liebigs. Ann. Chem. 426; 1922; 132) (5.0 g, 26 mmol) in toluene (300 ml) and methanol (100 ml) and the reaction mixture was stirred at room temperature for 72 hours. The mixture was evaporated under reduced pressure to afford the title compound as a solid, 4.94 g

¹H-nmr (CDCl₃,400MHz) δ: 1.65 (m, 4H), 2.64-2.80 (2m, 4H), 3.92 (s, 3H), 6.6 (d, 1H), 7.5 (d, 1H), 11.00 (s, 1H).

LRMS: m/z APCI⁺ 207 [MH⁺]

### PREPARATION 6

### Methyl 7-f[(trifluoromethyl)sulfonyl]oxy}-2-naphthoate

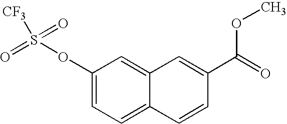

According to the method of preparation 5, the acid from preparation 4 (5.40 g, 28.7 mmol) was converted to methyl 7-hydroxy-2-naphthoate. This material was then converted to the title compound in 81 % yield by the method of preparation 1.

¹H-nmr (CD₃OD, 400 MHz) δ: 3.95 (m, 3H), 7.60 (d, 1H), 8.10 (m, 4H), 8.65 (s, 1H)

### PREPARATION 7

### Methyl 1-{[(trifluoromethyl)sulphonyl]oxy}-5,6,7,8-tetrahydronaphthalene-2-carboxylate

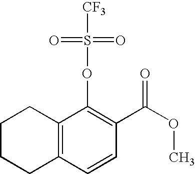

N-Phenyltrifluoromethanesulphonimide (50.59 g, 141 mmol), the alcohol from preparation 5 (13.25 g, 64 mmol) and 4-dimethylaminopyridine (catalytic) in dichloromethane (600 ml) was stirred at room temperature for 30 minutes. N-Ethyidiisopropylamine (24.5 ml, 141 mmol) was then added and the reaction was stirred at room temperature for 18 hours. The reaction was washed with 0.5M hydrochloric acid (×3), water, then brine, dried (MgSO₄) and evaporated under reduced pressure. The crude product was purified by column chromatography on silica gel using dichloromethane as eluant to afford the title compound in quantitative yield.

¹H-nmr (CDCl₃, 400 MHz) δ: 1.80 (d, 4H), 2.82 (d, 4H), 3.90 (s, 3H), 7.40-7.70 (2d, 2H).

LRMS m/z APCI⁺ 339 [MH⁺]

### PREPARATION 8

### Methyl 1-methyl-5,6,7,8-tetrahydronaphthalene-2-carboxylate

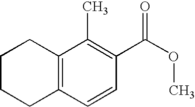

A mixture of the compound from preparation 7 (12.0 g, 35 mmol), trimethylboroxine (8.91 g, 71 mmol), tetrakis(triphenylphosphine)palladium (0) (8.2 g, 7.1 mmol) and potassium carbonate (9.79 g, 71 mmol) in dioxan (120 ml) was stirred at 110° C. for 18 hours. The cooled mixture was partitioned between ethyl acetate and water, and the layers were separated. The organic phase was washed with brine, dried (MgSO₄) and evaporated under reduced pressure. The residue was purified by column chromatography on silica gel using dichloromethane as eluant to give the title compound as a solid, 7.21 g.

¹H-nmr (CDCl₃, 400 MHz) δ: 1.73-1.90 (2×m, 4H), 2.40 (s, 3H), 2.65 (m, 2H), 2.80 (m, 2H), 3.85 (s, 3H), 6.95 (d, 1H), 7.53 (d, 1H).

LRMS m/z APCI⁺ 205 [MH⁺]

### PREPARATION 9

### 7-Methyl-2-naphthoic acid methyl ester

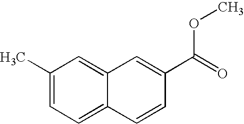

A mixture of the compound from preparation 6 (1.2 g, 3.6 mmol), trimethylboroxine (1.0 ml, 7.18 mmol), tetrakis(triphenylphosphine)palladium (0) (830 mg, 0.72 mmol) and potassium carbonate (2.0 g, 14.5 mmol) in dioxan (15 ml) was stirred at 110° C. for 18 hours. The cooled mixture was diluted with ethyl acetate, then filtered, and the filtrate was concentrated under reduced pressure. The residue was pre-adsorbed onto silica gel, and purified by column chromatography using an elution gradient of pentane:ethyl acetate (100:0 to 97:3) to afford the title compound as a white solid, 690 mg.

¹H-nmr (CDCl₃, 400 MHz) δ: 2.55 (s, 3H), 4.00 (s, 3H), 7.40 (d, 1H), 7.70 (s, 1H), 7.75 (d, 1H), 7.80 (d, 1H), 8.00 (d, 1H), 8.50 (s, 1H).

### PREPARATION 10

### 5-Methyl-2-naphthoic acid methyl ester

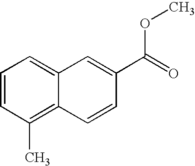

Trimethyl boroxine (1.26 ml, 9.04 mmol) was added to a suspension of methyl 5-bromo-naphthalene-2-carboxylate (Helv. Chim. Acta. 21; 1938; 62) (1.2 g, 4.52 mmol), potassium carbonate (2.5 g, 18.08 mmol) and tetrakis(triphenylphosphine)palladium (0) (1.04 g, 0.9 mmol) in dioxan (20 ml), and the reaction was heated under reflux for 2.5 hours. The cooled reaction was partitioned between ethyl acetate (100 ml) and water (100 ml), and the layers were separated. The organic phase was dried (MgSO₄) and evaporated under reduced pressure. The residual orange oil was purified by column chromatography on silica gel using an elution gradient of pentane:ethyl acetate (100:0 to 98:2) to give the title compound as an oil, 850 mg.

¹H-nmr (CDCl₃, 400 MHz) δ: 2.72 (s, 3H), 4.00 (s, 3H), 7.43 (s, 2H), 7.80 (m, 1H), 8.04 (m, 2H), 8.60 (s, 1H).

### PREPARATION 11

### 1-Methyl-5,6,7,8-tetrahydronaphthalene-2-carboxylic acid

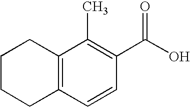

A solution of the ester from preparation 8 (7.20 g, 35 mmol) and 2N sodium hydroxide (35 ml) in tetrahydrofuran (130 ml) was stirred under reflux for 18 hours. The mixture was neutralised using 2N hydrochloric acid, and extracted with dichloromethane (3×). The combined organic solutions were dried (MgSO₄), and evaporated under reduced pressure. The crude product was purified by column chromatography on silica gel using an elution gradient of dichloromethane:methanol (100:0 to 97:3) to afford the title compound as a solid, 3.11 g.

¹H-nmr (CDCl₃, 400 MHz) δ: 1.80 (m, 2H), 2.89 (m, 2H), 2.52 (s, 3H), 2.70 (t, 2H), 2.83 (t, 2H), 7.00 (d, 1H), 7.70 (d, 1H), 11.90 (s, 1H).

LRMS m/z APCI⁺ 191 [MH⁺]

### PREPARATION 12

### 7-Methyl-2-naphthoic acid

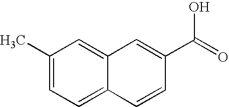

A mixture of the ester from preparation 9 (680 mg, 3.4 mmol) and lithium hydroxide (490 mg, 20.4 mmol) in tetrahydrofuran (9 ml) and water (2 ml) was heated under reflux for 18 hours. The cooled mixture was acidified to pH 1 using 6N hydrochloric acid; and the resulting white precipitate was filtered off, washed with water and dried in vacuo at 50° C. to afford the title compound, 520 mg.

¹H-nmr (DMSO-d₆, 400 MHz) δ: 2.50 (s, 3H), 7.45 (d, 1H), 7.90 (m, 4H), 8.45 (s, 1H).

LRMS: m/z (APCI⁻) 185 [M−H⁻]

### PREPARATION 13

### 5-Methyl-2-naphthoic acid

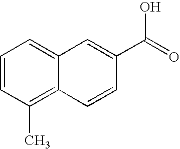

A solution of lithium hydroxide (254 mg, 10.6 mmol) in water (6 ml) was added dropwise to a solution of the ester from preparation 10 (850 mg, 4.25 mmol) in tetrahydrofuran (22 ml) and the reaction mixture was stirred at room temperature for 18 hours. The reaction was then warmed to 60° C. for a further 3 hours, cooled, and the solution was acidified to pH 1 using 2N hydrochloric acid. The mixture was diluted with water, and the resulting precipitate was filtered off and dried in vacuo to afford the title compound as a white solid, 713 mg.

¹H-nmr (DMSO-d₆, 400 MHz) δ: 2.65 (s, 3H), 7.44 (s, 2H), 7.96 (m, 1H), 8.00 (d, 1H), 8.05 (d, 1H), 8.58 (s, 1H).

LRMS: m/z APCI⁺ 214 [MH⁺]

### PREPARATION 14

### Methyl 7-amino-naphthalene-2-carboxylate

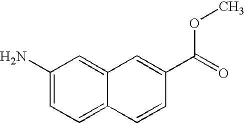

A mixture of the triflate from preparation 6 (3.27 g, 9.8 mmol), palladium acetate (210 mg, 0.98 mmol), benzophenone imine (1.69 ml, 14 mmol), (±)-BINAP (700 mg, 0.98 mmol) and caesium carbonate (4.10 g, 13 mmol) in tetrahydrofuran (50 ml) was heated under reflux for 18 hours. The cooled mixture was filtered through a pad of silica gel and the filtrate was concentrated under reduced pressure. The solid was recrystallised from methanol to give methyl 7-(benzhydrylidene-amino)-naphthalene-2-carboxylate This was dissolved in methanol, palladium hydroxide (10% wt) was added and the mixture was hydrogenated at 50 psi and room temperature. The reaction mixture was filtered through Arbocel®, the filtrate was evaporated under reduced pressure and the residue was triturated with pentane (3×50 ml), to give the title compound as a yellow-brown solid, 1.43 g.

¹H-nmr (CD₃OD, 400 MHz) δ: 3.85 (s, 3H), 6.85 (m, 2H), 7.05 (m, 2H), 7.60 (m, 2H)

LRMS: m/z APCI⁺ 202 [MH⁺]

### PREPARATION 15

### 7-Fluoro-2-naphthoic acid

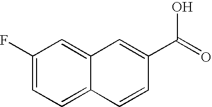

The amine from preparation 14 (1.43 g, 7.1 mmol) was added to 1-ethyl-3-methyl-1H-imidazolium tetrafluoroborate (10 ml), and stirred at room temperature for 1 hour. Nitrosonium tetrafluoroborate (840 mg, 7.2 mmol) was added portionwise over 30 minutes, and once addition was complete, the reaction was stirred at room temperature for 18 hours under nitrogen. The reaction was then warmed slowly to 90° C., and stirred for a further 6 hours. The cooled reaction was added to a mixture of lithium hydroxide (1 g) in tetrahydrofuran (25 ml) and this suspension was stirred for 18 hours. Concentrated hydrochloric acid was carefully added to adjust the pH to 1, and the mixture was cooled in the fridge for 2 hours. Water (50 ml) was added, and the resulting precipitate was filtered off and dried in vacuo, to afford the title compound as a white solid, 1.12 g.

¹H-nmr (DMSO, 400 MHz) δ: 7.40 (t, 1H), 7.70 (m, 2H), 8.10 (d, 1H), 8.20 (t, 1H), 9.00 (d, 1H)

LRMS: m/z APCI³¹, 189 [M−H]⁻

### PREPARATION 16

### tert-Butyl 4-{[(2,4-dinitrophenyl)sulfonyl]amino}piperidine-1-carboxylate

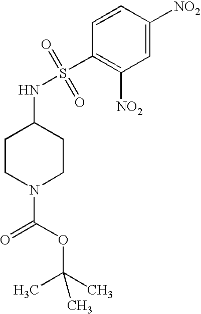

Lutidine (1.16 ml, 9.9 mmol) and a solution of 2,4-dinitrosulphonyl chloride (1.26 g, 4.7 mmol) in dichloromethane (30 ml) was added to an ice-cooled solution of 4-amino-piperidine-1-carboxylate (1.0 g, 5.0 mmol) in dichloromethane (70 ml), and the resulting solution was stirred at 0° C. for 30 minutes. The solution was allowed to warm to room temperature and then stirred for a further 18 hours. Water (100 ml) was added, the mixture was stirred for 30 minutes, then basified to pH 2.5-3 using 5% aqueous potassium hydrogen sulphate solution. The phases were separated, the organic layer was washed with water (2×100 ml), brine (100 ml), dried (MgSO₄) and evaporated under reduced pressure to give the title compound as a light yellow solid in quantitative yield.

¹H-nmr (CD₃OD, 400 MHz) δ: 1.45 (s, 11H), 1.80 (m, 2H), 2.85 (m, 2H), 3.50 (m,1H), 3.95 (m, 2H), 8.35 (d, 1H), 8.60 (d, 1H), 8.70 (s, 1H)

LRMS: m/z APCI⁻ [M−H]⁻ 429

### PREPARATION 17

### tert-Butyl 4-(cvclobutylmethyl-amino)-piperidine-1-carboxylate

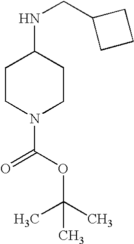

Cyclobutanemethanol (0.77 ml, 8.1 mmol) and triphenylphosphine (2.34 g, 9 mmol) were added to a solution of the compound from preparation 16 (3.20 g, 7.4 mmol) in tetrahydrofuran (100 ml), and this solution was cooled to 0° C. Diisopropyl azodicarboxylate (1.80 g, 9 mmol) was added dropwise over 30 minutes, so as to maintain the internal temperature at 0° C., and the mixture was then stirred for a further 30 minutes at 0° C., before being allowed to warm to room temperature. The mixture was concentrated under reduced pressure, the residue was dissolved in dichloromethane (200 ml), mercaptoacetic acid (0.67 ml, 9.6 mmol) and triethylamine were added and the reaction was stirred at room temperature for 30 minutes. A solution of potassium hydrogensulphate (3.62 g) in water (100 ml) was added and the mixture was stirred at room temperature for 1 hour. The pH of the solution was adjusted to 2.5-3.0 by the addition of 2% aqueous potassium hydrogen sulphate solution, and then the layers were separated. The aqueous phase was washed with ethyl acetate (3×), basified to pH 12 using 2N sodium hydroxide solution, then extracted with ethyl acetate (3×50 ml). The combined organic extracts were dried (MgSO₄), and evaporated under reduced pressure to afford the title compound as a colourless oil, 1.76 g.

¹H-nmr (CD₃OD, 400 MHz) δ: 1.25 (m, 2H), 1.45 (s, 9H), 1.70 (m, 2H), 1.95 (m, 4H), 2.10 (m, 2H), 2.45 (m, 1H), 2.60 (m, 3H), 2.80 (m, 2H), 4.05 (d, 2H).

LRMS: m/z APCI⁺ 269 [MH⁺]

### PREPARATION 18

### 4,4,4-Trifluorobutyl-1-amine hydrochloride

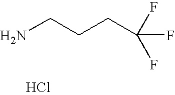

A solution of diethylazodicarboxylate (11.2 ml, 69 mmol) in toluene (50 ml) was added slowly to a solution of 4,4,4-trifluorobutanol (9.05 g, 69 mmol), triphenylphosphine (18.34 g, 69 mmol) and di-tert-butyl iminodicarboxylate (14.0 g, 63 mmol) in toluene (100 ml), so as to maintain the reaction temperature between 20-25° C. The reaction was then stirred at room temperature for 18 hours. Trifluoroacetic acid (50 ml) was added dropwise to the ice-cooled mixture and, once addition was complete, the reaction was allowed to warm to room temperature and stirred for 24 hours. The reaction was diluted with water (150mi), and the layers were separated. The organic phase was extracted with water (2×150 ml), and the combined aqueous layers were washed with ether (2×100 ml), then carefully basified by the addition of 5N sodium hydroxide solution. The aqueous was extracted with ether, these combined extracts were dried (MgSO₄), and treated with 2N HCl in ether, and the solution was left at room temperature for 18 hours. The solution was concentrated under reduced pressure, the residue was azeotroped with toluene, triturated with ether, and recrystallised from acetone/ether to give the title compound, 4.17 g.

¹H-nmr (DMSO-d₆, 400 MHz) δ: 1.80 (m, 2H), 2.39 (m, 2H), 2.82 (t, 2H), 8.20 (brs, 3H).

### PREPARATION 19

### tert-Butyl (3R)-1-(trifluoroacetyl)pyrrolidin-3-ylcarbamate

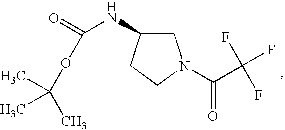

(3R)-3-(tert-Butoxycarbonylamino)pyrrolidine (3.0 g, 16.1 mmol) and pyridine (3.87 ml, 48.3 mmol) were dissolved in dichloromethane (55 ml) and the reaction mixture was stirred under nitrogen at 0° C. for 1 hour. A solution of trifluoroacetic acid anhydride (2.74 ml, 32.2 mmol) in dichloromethane (5 ml) was added dropwise to the reaction mixture over 10 minutes. The reaction mixture was allowed to warm to room temperature and stirred for 2 hours. The reaction mixture was diluted with dichloromethane (100 ml) and washed with saturated sodium hydrogencarbonate solution, water and then brine. The organic layer was separated, dried (MgSO₄) and concentrated in vacuo. The crude product was azeotroped with toluene (2×30 ml) to yield the title product.

¹HNMR(DMSO-d₆, 400 MHz) δ: 1.40 (s, 9H), 1.82 (dd, 1H), 2.08 (dd, 1H), 3.33 (m, 1H), 3.46 (m, 1H), 3.59-3.77 (brm, 2H), 4.06 (m, 1H), 7.22 (m,1H)

LRMS ESI+m/z 281 [MH]⁺

### PREPARATION 20

### (3R)-1-(trifluoroacetyl)pyrrolidin-3-amine hydrochloride

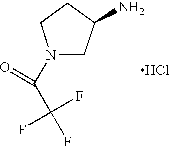

The Boc protected amine of preparation 19 (4.59 g, 16.1 mmol) was dissolved in dichloromethane (100 ml) and the reaction mixture was stirred at 0° C. for 1 hour. Hydrogen chloride gas was then bubbled through the solution for 10 minutes and the reaction mixture was allowed to warm to room temperature. Hydrogen chloride gas and subsequently nitrogen gas were then bubbled through the solution for 15 and 10 minutes respectively and the reaction mixture was then concentrated in vacuo to yield the title product as a pale yellow crystalline solid.

¹H-nmr (CDCl₃, 400 MHz) δ: 1.27 (m, 2H), 1.65 (m, 1H), 1.81 (m, 1H), 2.10 (m, 2H), 3.32 (m, 2H), 3.61 (m, 1H)

LRMS ES⁺ m/z 183 [MH]⁺

### PREPARATION 21

### (3R)-N-Isopropyl-1-(trifluoroacetyl)pyrrolidin-3-amine

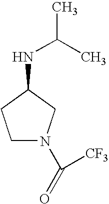

A mixture of the amine from preparation 20 (2.0 g, 8.31 mmol), triethylamine (1.16 ml, 8.31 mmol), and 10% palladium on charcoal (200 mg) in acetone (10 ml), was hydrogenated at 60 psi and room temperature for 18 hours. The mixture was filtered through Arbocel®, washing through with acetone, and the filtrate was evaporated under reduced pressure. The crude product was purified by column chromatography on silica gel using an elution gradient of dichloromethane:methanol (100:0 to 95:5) to afford the title compound as an oil, 1.22 g.

¹H-nmr (CDCl₃, 400 MHz) δ: 1.00-1.20 (m, 6H), 1.60-1.90 (m, 2H), 2.00-2.20 (m, 1H), 2.80-2.90 (m, 1H), 3.20-3.90 (m, 5H).

LRMS: m/z (APCI⁺) 225 [MH⁺]

### PREPARATION 22

### (3R)-N-Isobutyl-1-(trifluoroacetyl)pyrrolidin-3-amine

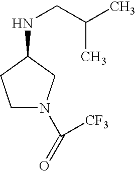

A mixture of the amine from preparation 20 (500 mg, 2.29 mmol), isobutyraldehyde (212 μl, 2.33 mmol), triethylamine (318 μl, 2.29 mmol) and 10% palladium on charcoal (60 mg) in ethanol (10 ml), was hydrogenated at 60 psi and room temperature for 18 hours. The mixture was filtered through Arbocel®, washing through with ethyl acetate, and the filtrate was evaporated under reduced pressure. The residue was triturated with ethyl acetate, the precipitate was filtered off, and the filtrate was washed with a small amount of aqueous sodium bicarbonate solution. The organic layer was separated, dried (MgSO₄) and evaporated under reduced pressure to afford the title compound as a yellow oil, 320 mg.

¹H-nmr (CDCl₃, 400 MHz) δ: 0.97 (2×d, 6H), 1.73-2.58 (m, 5H), 3.41-3.98 (m, 5H)

LRMS: m/z APCI⁺ 239 [MH⁺]

### PREPARATION 23

### (3S)-N-Isobutyl-1-(trifluoroacetyl)pyrrolidin-3-amine

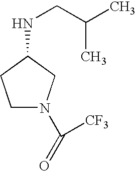

The title compound was obtained in 85% yield as a brown oil, from (S)-1-(trifluoroacetyl)pyrrolidin-3-yl amine (J. Med. Chem., 1996, 39(14), pg. 2771), and isobutyraldehyde, following the procedure described in preparation 22.

¹H-nmr (CDCl₃, 400 MHz) δ: 0.90 (d, 6H), 1.59-1.96 (m, 3H), 2.42 (m, 2H), 3.34-3.47 (m, 2H), 3.56-3.89 (m, 3H)

LRMS: m/z APCI⁺ 239 [MH⁺]

### PREPARATION 24

### (3R)-N-Butyl-1-(trifluoroacetyl)pyrrolidin-3-amine

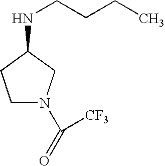

A mixture of the amine from preparation 20 (2.0 g, 8.3 mmol), butyraldehyde (750 μl, 8.3 mmol), triethylamine (1.16 ml, 8.3 mmol), acetic acid (4 ml) and sodium triacetoxyborohydride (2.11 g, 9.96 mmol) in dichloromethane (40 ml) was stirred at room temperature for 18 hours. The mixture was quenched by the addition of aqueous sodium bicarbonate solution, the layers were separated, and the aqueous phase was extracted with ethyl acetate (2×100 ml). The combined organic layers were evaporated under reduced pressure and the residue was purified by column chromatography on silica gel using an elution gradient of pentane:ethyl acetate:triethylamine (75:25:0 to 0:100:0 to 0:99:1) to afford the title compound, 1.31 g.

¹H-nmr (CDCl₃, 400 MHz) δ: 0.88 (t, 3H), 1.35 (m, 2H), 1.42 (m, 2H), 1.76, 1.84 (2×m, 1H), 2.08 (m, 1H), 2.60 (m, 2H), 3.40 (m, 2H), 3.55-3.80 (m, 3H).

LRMS: m/z APCI⁺ 239 [MH⁺]

### PREPARATIONS 25 TO 28

The following compounds of general formula:

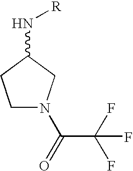

were prepared from the amine from preparation 20 or (S)-1-(trifluoroacetyl)pyrrolidin-3-amine (J. Med. Chem. 1996, 39, 2764) and the appropriate aldehyde.

| Prep | | | |
|---|---|---|---|
| No | Stereo | R | Data |
| 25¹ | R | 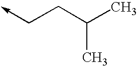 | ¹H-nmr (CDCl₃ 400 MHz) δ: 0.9 (d, 6H), 1.3-1.4 (q, 2H), 1.6 (m, 1H), 1.7-1.9 (m, 1H), 2.1 (m, 1H), 2.6 (m, 2H), 3.4 (m, 2H), 3.6-3.8 (m, 3H) LRMS: m/z APCI⁺ 253 [MH⁺] |
| 26 | R | 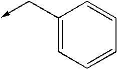 | ¹H-nmr (CDCl₃, 400 MHz) (rotamers) δ: 1.70-2.15 (m, 2H), 3.40-3.50 (m, 2H), 3.50-3.80 (m, 1H), 3.50-3.80 (2xm, 2H), 3.80 (s, 2H), 7.25-7.35 (m, 5H) LRMS: m/z ES⁺ 274 [MH⁺] |
| 27¹ | R | 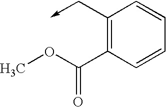 | ¹H-nmr (CDCl₃ 400 MHz) δ: 2.2-2.4 (m, 2H), 3.6 (s, 3H), 3.6-3.8 (m, 1H), 3.8-4.1 (m, 3H), 4.4 (d, 2H), 5.0-5.1 (m, 1H), 7.4-7.6 (m, 4H), 7.8-7.9 (m, 1H) |
| 28 | S | 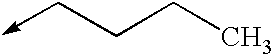 | ¹H-nmr (CDCl₃ , 400 MHz) δ 0.95 (t, 3H), 1.35 (m, 2H), 1.50 (m, 2H), 1.85 (m, 1H), 2.15 (m, 1H), 2.65 (m, 2H), 3.45 (m, 2H), 3.70 (m, 3H) LRMS: m/z (APCI⁺) = 239 [MH⁺] |

¹= product purified using dichloromethane:methanol:0.88 ammonia as column eluant

### PREPARATION 29

### tert-Butyl 4-(neopentylamino)piperidine-1-carboxylate

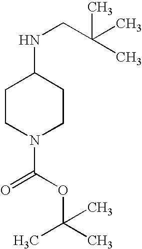

2,2-Dimethylpropylamine (460 mg, 5.28 mmol) was added to a solution of tert-butyl 4-oxopiperidine-1-carboxylate (1.0 g, 5.01 mmol) in methanol (15 ml) and the solution was stirred at room temperature for 24 hours. The solution was cooled in an ice-bath, and sodium borohydride (380 mg, 10.0 mmol) was added portionwise. The reaction mixture was stirred at room temperature for 18 hours, then water was added carefully to quench the reaction. The mixture was partitioned between ethyl acetate (100 ml) and brine (80mi), the layers were separated, and the organic phase was dried (MgSO₄) and concentrated under reduced pressure. The residue was purified by column chromatography on silica gel using an elution gradient of ethyl acetate:pentane (50:50 to 100:0). The product was repurified by column chromatography, eluting with ethyl acetate:pentane (0:100 to 30:70) to afford the title compound as a colourless oil, 760 mg.

¹H-nmr (CDCl₃, 400 MHz) δ 0.91 (s, 9H), 1.23-1.33 (br, M, 2H), 1.45 (s, 9H), 1.82-1.85 (br, m, 2H), 2.37 (s, 2H), 2.58 (br, m, 1H), 2.76-2.84 (br, m, 2H), 4.00 (br, m, 2H).

LRMS: m/z APCI⁺: 271 [MH⁺], 215 [MH⁺−t-Bu], 272 [MH⁺−Boc].

### PREPARATION 30

### tert-Butyl 4-(cyclopentylmethylamino)piperidine-1-carboxylate

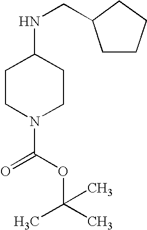

Triethylamine (9.2 ml, 66 mmol) was added to a solution of cyclopentylmethylamine hydrochloride (J.Med.Chem. 40; 20; 1997; 3207) (6.0 g, 44 mmol) and tert-butyl 4-oxopiperidine-1-carboxylate (8.77 g, 44 mmol) in methanol (110 ml), and the solution was stirred at room temperature for 18 hours. Sodium borohydride (3.3 g, 88 mmol) was added portionwise, and the reaction mixture was stirred for a further 3 hours. The mixture was partitioned between sodium bicarbonate solution and ethyl acetate/methanol, and the layers were separated. The organic phase was dried (MgSO₄) and evaporated under reduced pressure. The residual oil was purified by column chromatography on silica gel using an elution gradient of dichloromethane:methanol:0.88 ammonia (100:0:0 to 90:10:1) to afford the title compound, 8.46 g.

¹H-nmr (CD₃OD, 400 MHz) δ: 1.10-1.30 (m, 4H), 1.45 (s, 9H), 1.50-1.69 (m, 4H), 1.75-1.92 (m, 4H), 1.92-2.07 (m, 1H), 2.63 (d, 2H), 2.57-2.68 (m, 1H), 2.71-2.84 (brm, 2H), 4.07 (d, 2H)

LRMS: m/z APCI⁺ 283 [MH⁺]

### PREPARATION 31

### tert-Butyl 4-[(4-hydroxybutyl)aminolpiperidine-1-carboxylate

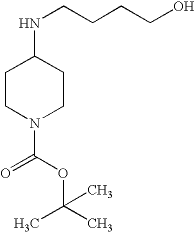

A solution of 4-amino-1-butanol (10.3 ml, 112 mmol) and tert-butyl 4-oxopiperidine-1-carboxylate (11.2 g, 56 mmol) in methanol (225mi) was stirred at room temperature for 4 hours. Sodium borohydride (4.2 g, 112 mmol) was added portionwise, and the reaction was stirred for a further 18 hours. The mixture was quenched by the addition of sodium bicarbonate solution and then partitioned between ethyl acetate (750 ml) and water (750 ml). The layers were separated, the organic phase was dried (MgSO₄) and evaporated under reduced pressure to afford the title compound as a clear oil, 12.4 g.

¹H-nmr (CDCl₃, 400 MHz) δ: 1.19-1.35 (m, 1H), 1.44 (s, 9H), 1.67 (m, 4H), 1.88 (bd, 2H), 2.57-2.83 (bm, 5H), 3.78 (t, 2H), 4.04 (m, 2H)

LRMS: m/z APCI⁺ 273 [MH⁺]

### PREPARATION 32

### tert-Butyl 4-[(4,4,4-trifluorobutyl)amino]piperidine carboxylate

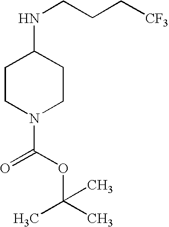

Triethylamine (1.06 ml, 7.5 mmol) was added dropwise to a solution of the amine hydrochloride from preparation 18 (1.23 g, 7.5 mmol) and tert-butyl 4-oxopiperidine-1-carboxylate (1.5 g, 7.5 mmol) in dichloromethane (25 ml). Acetic acid (2 ml) was then added, followed by sodium triacetoxyborohydride (portionwise) (1.67 g, 7.5 mmol) and the reaction mixture was stirred at room temperature for 18 hours. The reaction mixture was poured into water, basified using sodium bicarbonate, and then extracted using dichloromethane. The combined organic extracts were evaporated under reduced pressure and the residue was purified by column chromatography on silica gel using an elution gradient of dichloromethane:methanol (100:0 to 95:5) to afford the title compound as an oil, 2.07 g.

1H-nmr (CDCl₃, 400 MHz) δ: 1.23 (m, 2H), 1.47 (s, 9H), 1.73 (m, 2H), 1.82 (m, 2H), 2.18 (m, 2H), 2.60 (m, 1H), 2.70 (m, 2H), 2.80 (m, 2H), 4.01 (m, 2H).

LRMS: m/z APCI⁺ 279 [MH⁺]

### PREPARATION 33

### tert-Butyl 4-(n-pentylamino)piperidine-1-carboxylate

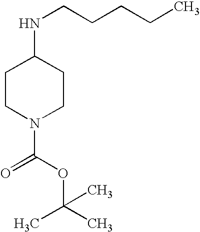

The title compound was obtained as a clear oil in 92% yield from tert-butyl 4-oxopiperidine-1-carboxylate and 1-aminopentane, following the procedure described in preparation 32.

¹H-nmr (CDCl₃, 400 MHz) δ: 0.95 (t, 3H), 1.15-1.35 (m, 6H),1.40-1.50 (m, 12H), 1.80 (d, 2H), 2.55 (m, 3H), 2.75 (q, 2H), 4.00 (br, 2H).

LRMS: m/z APCI⁺ 271 [MH⁺]

### PREPARATION 34

### tert-Butyl 4-(cyclopentylamino)piperidine-1-carboxylate

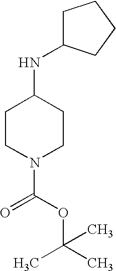

A solution of cyclopentanone (139 mg, 1.65 mmol) and tert-butyl 4-amino-piperidine-1-carboxylate (300 mg, 1.5 mmol) in methanol (6 ml) was stirred at room temperature for 18 hours. Sodium borohydride (1 13 mg, 3.0 mmol) was added portionwise, and the reaction mixture was stirred for a further 6 hours. The mixture was partitioned between saturated aqueous sodium bicarbonate solution and ethyl acetate and the layers were separated. The aqueous layer was extracted with further ethyl acetate (×3) and the combined organic extracts were dried (MgSO₄) and evaporated under reduced pressure. The residue was purified by column chromatography on silica gel using an elution gradient of pentane:ethyl acetate:methanol (50:50:0 to 0:90:10) to give the title compound as a colourless oil, 310 mg.

¹H-nmr (CDCl₃, 400 MHz) δ: 1.22-1.33 (m, 4H), 1.45 (s, 9H), 1.49-1.58 (m, 2H), 1.64-1.73 (m, 2H), 1.84-1.94 (m, 4H), 2.63-2.70 (m, 1H), 2.71-2.80 (brm, 2H), 3.21 (q, 1H), 4.05 (brm, 2H).

LRMS: m/z APCI⁺ 269 [MH⁺]

### PREPARATION 35

### tert-Butyl 4-(cyclobutylamino)piperidine-1-carboxylate

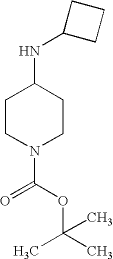

The title compound was obtained as a colourless oil in 58% yield from cyclobutanone and tert-butyl 4-amino-piperidine-1-carboxylate, following the procedure described in preparation 34.

¹H-nmr (CDCl₃, 400 MHz) δ: 1.21-1.29 (m, 2H), 1.45 (s, 9H), 1.59-1.80 (m, 6H), 2.19-2.26 (m, 2H), 2.58-2.65 (m, 1H), 2.69-2.78 (brm, 2H), 3.32-3.39 (m, 1H), 4.02 (brm, 2H).

LRMS: m/z APCI⁺ 255 [MH⁺]

### PREPARATION 36

### tert-Butyl 4-(isopropylamino)piperidine-1-carboxylate

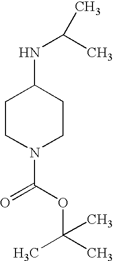

The title compound was obtained from acetone and tert-butyl 4-amino-piperidine-1-carboxylate, following the procedure described in preparation 34.

¹H-nmr (CDCl₃, 400 MHz) δ 1.07 (d, 6H), 1.18-1.29 (m, 2H), 1.45 (s, 9H), 1.84-1.87 (m, 2H), 2.68-2.80 (m, 3H), 2.95-3.05 (m, 1H), 4.06 (br, m, 2H).

LRMS: APCI⁺: 243 [MH⁺], 187 [MH⁺−t-Bu], 143 [MH⁺−Boc]

### PREPARATION 37

### tert-Butyl 4-(ethylamino)piperidine-1-carboxylate

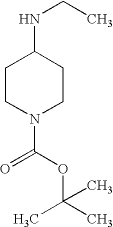

The title compound was obtained as a colourless oil in 96% yield from tert-butyl 4-oxopiperidine-1-carboxylate and ethylamine, following the procedure described in preparation 32.

¹H-nmr (CDCl₃, 400 MHz) δ 1.11 (t, 3H), 1.21-1.31 (m, 2H), 1.44 (s, 9H), 1.81-1.85 (m, br, 2H), 2.59-2.66 (m, 1H), 2.69 (q, 2H), 2.76-2.83 (m, 2H), 3.99-4.02 (m, br, 2H).

LRMS: APCI⁺ 229 [MH⁺]

### PREPARATION 38

### tert-Butyl 4-(2-methylpropylamino)piperidine-1-carboxylate

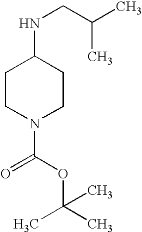

The title compound was obtained as a colourless oil in 84% yield from isobutyraldehyde and tert-butyl 4-amino-piperidine-1-carboxylate, following the procedure described in preparation 34.

¹H-nmr (CDCl₃, 400 MHz) δ 0.91 (d, 6H), 1.21-1.30 (m, 2H), 1.45 (s, 9H), 1.66-1.76 (m, 1H), 1.81-1.85 (m, br, 2H), 2.43 (d, 2H), 2.54-2.61 (m, 1H), 2.75-2.81 (m, br, 2H), 4.02 (br, 2H).

LRMS: APCI⁺: 257 [MH⁺]

### PREPARATION 39

### tert-Butyl 4-(cyclopropylmethylamino)piperidine-1-carboxylate

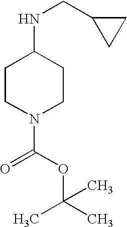

The title compound was obtained from cyclopropanemethanol and the sulfonamide from preparation 16 following the procedure described in preparation 17.

¹H-nmr (CD₃OD, 400 MHz) δ: 0.15 (m, 2H), 0.50 (m, 2H), 0.90 (m, 1H), 1.25 (m, 2H), 1.45 (s, 9H), 1.85 (m, 2H), 2.45 (d, 2H), 2.65 (m, 1H), 2.80 (m, 2H), 4.05 (m, 2H), 8.2 (d, 1H)

LRMS: m/z APCI⁺ 255 [MH⁺]

### PREPARATION 40

### tert-Butyl 4-[butyl-(3-chloro-2-methyl-benzoyl)-amino]-piperidine-1-carboxylate

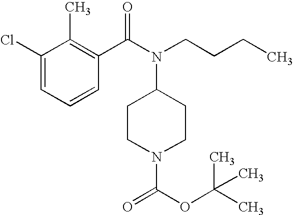

A mixture of 3-chloro-2-methyl-benzoic acid (200 mg, 1.12 mmol), 1-(tert-butoxycarbonyl)-4-(butylamino)piperidine (WO 09618628, ex 86) (300 mg, 1.12 mmol), 1-hydroxybenzotriazole hydrate (221 mg, 1.64 mmol), 1-(3-dimethylaminopropyl)-3-ethylcarbodiimide hydrochloride (313 mg, 1.64 mmol) and triethylamine (325 μl, 2.34 mmol) in dichloromethane (10 ml) was stirred at room temperature for 18 hours. The mixture was washed with saturated sodium bicarbonate solution, and evaporated under reduced pressure. The residue was purified by column chromatography on silica gel using an elution gradient of pentane:dichloromethane:methanol (50:50:0 to 0:100:0 to 0:95:5) to afford the title compound, 243 mg.

¹H-nmr (CDCl₃, 400 MHz) rotamers δ: 0.80 (m, 2H), 0.95 (m, 3H), 1.30-1.80 (m, 15H), 2.20 (2×s, 3H), 2.80-3.40 (m, 4H), 4.00-4.50 (m, 3H), 7.00 (m, 1H), 7.10 (m, 1H), 7.30 (m, 1H).

LRMS: m/z APCI⁺ 409 [MH⁺]

### PREPARATION 41

### tert-Butyl-4-[butyl-(1-napthoyl)amino]piperidine-1-carboxylate

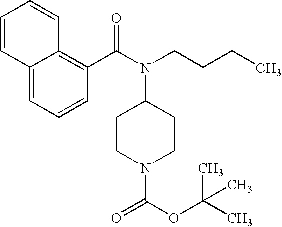

1-Napthoyl chloride (0.1 ml, 0.66 mmol) was added to a solution of tert-butyl 4-(butylamino)piperidine-1-carboxylate (WO 09618628, ex 86) (154 mg, 0.6 mmol) and triethylamine (0.25 ml, 1.8 mmol) in dichloromethane (6 ml), and the reaction mixture was stirred at room temperature for 4 hours. The solution was evaporated under reduced pressure and the residue was purified by column chrmatography on silica gel using a Varian Bond Elut® cartridge and an elution gradient of pentane:ethyl acetate (100:0 to 50:50) to afford the title compound as a colourless oil, 233 mg.

¹H-nmr (CDCl₃, 400 MHz) δ rotamers: 0.52, 1.03 (2×t, 3H), 1.41, 1.50 (2×s, 9H), 0.84-4.05 (m, 15H), 7.33-7.39 (m, 1H), 7.44-7.51 (m, 3H), 7.72-7.81 (m, 1H), 7.83-7.87 (m, 2H).

LRMS: m/z APCI⁺ 411 [MH⁺]

### PREPARATION 42

### tert-Butyl-4-[neopentyl(2-napthoyl)amino]piperidine-1-carboxylate

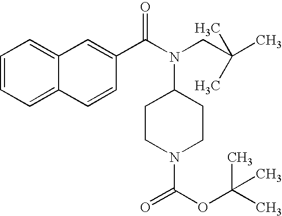

A solution of the amine from preparation 29 (235 mg, 0.87 mmol) in dichloromethane (2 ml) and triethylamine (325 μl, 2.34 mmol) was added dropwise to a suspension of 2-naphthoyl chloride (150 mg, 0.78 mmol) in dichloromethane (5 ml), and the reaction mixture was stirred at room temperature for 18 hours. The mixture was partitioned between water (20 ml) and dichloromethane (30 ml) and the layers were separated. The organic phase was dried (MgSO₄) and evaporated under reduced pressure. The crude product was purified by column chromatography using a Bond Elut® cartridge and ethyl acetate:pentane (0:100 to 30:70) as eluant to give the title compound as a colourless oil, 251 mg.

¹H-nmr (CDCl₃, 400 MHz) δ: 1.05 (br, 9H), 1.45 (s, 9H), 1.70 (br, 4H), 2.40 (br, 2H), 3.30 (br, 2H), 3.95 (br, 1H), 4.15 (br, 2H), 7.55 (m, 3H), 7.90 (m, 3H), 7.95 (br, 1H)

LRMS: m/z (APCI⁺) 369 [MH⁺−^{t}Bu+H⁺]

### PREPARATION 43

### tert-Butyl 4-[cyclobutyl(2-naphthoyl)amino]piperidine-1-carboxylate

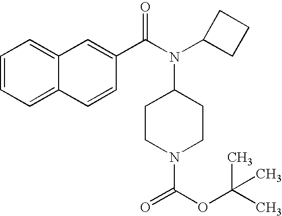

2-Naphthoyl chloride (0.1 g, 0.52 mmol) was added to a solution of the amine from preparation 35 (111 mg, 0.44 mmol) and triethylamine (0.18 ml, 1.29 mmol) in dichloromethane (4 ml), and the reaction mixture was stirred at room temperature for 22 hours. The reaction was quenched by the addition of ethanol (1 ml), and the solution was then evaporated under reduced pressure. The residue was purified by column chromatography on silica gel using a Bond Elut® cartridge and an elution gradient of pentane: ethyl acetate (100:0 to 50:50) to afford the the title compound as a white foam, 163 mg.

¹H-nmr (CDCl₃, 400Mhz) δ rotamers: 1.45-1.73 (m, 13H), 2.04-2.11 (m, 2H), 2.47 (br, 4H), 2.69 (br, 2H), 3.54-3.62 (m, 1H), 4.12-4.25 (m, 3H), 7.43 (dd, 1H), 7.51-7.55 (m, 2H), 7.83-7.87 (m, 4H).

LRMS: m/z APCI⁺ 409 [MH⁺]

### PREPARATION 44

### tert-Butyl 4-[cyclopentyl(2-naphthoyl)aminolpiperidine-1-carboxylate

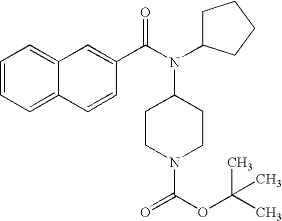

2-Naphthoyl chloride (165 mg, 0.87 mmol) was added to a solution of the amine from preparation 34 (155 mg, 0.58 mmol) and triethylamine (0.24 ml, 1.74 mmol) in dichloromethane (6 ml), and the reaction was stirred at room temperature for 18 hours. The reaction was quenched by the addition of saturated sodium bicarbonate solution, and the mixture was extracted with ethyl acetate (3×). The combined organic extracts were dried (MgSO₄) and evaporated under reduced pressure. The residue was purified by column chromatography on silica gel using a Varian Bond Elut® cartridge and an elution gradient of pentane: ethyl acetate (95:5 to 50:50) to afford the title compound as a colourless oil, 245 mg.

¹H-nmr (CDCl₃, 400 MHz) δ rotamers: 1.42-2.69 (m, 23H), 3.20-4.29 (m, 4H), 7.40 (dd, 1H), 7.49-7.54 (m, 2H), 7.80 (s, 1H), 7.84-7.86 (m, 3H).

LRMS: m/z APCI⁺ 423 [MH⁺]

### PREPARATION 45

### tert-Butyl 4-rcyclobutylmethyl(2-naphthoyl)aminolpiperidine-1-carboxylate

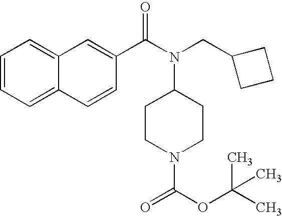

The amine from preparation 17 (170 mg, 0.62 mmol) and N-ethyldiisopropylamine (0.11 ml, 0.63 mmol) were added to a solution of 2-naphthoyl chloride (100 mg, 0.52 mmol) in dichloromethane (50 ml). 4-Dimethylaminopyridine (50 mg) was added and the solution was stirred at room temperature for 18 hours. The mixture was washed with 10% citric acid solution (3×), and then dried (MgSO₄) and evaporated under reduced pressure to give the title compound, 221 mg.

¹H-nmr (CD₃OD, 400 MHz) δ: Rotamers: 1.45 (s, 9H), 1.5-2.80 (br, 9H), 3.40-3.55 (br, 4H), 3.70-4.3 (br, 5H), 7.40 (d, 1H), 7.65 (m, 2H), 7.85 (s, 1H) (m, 3H).

LRMS: m/z (ES⁺) [M+Na⁺] 445

### PREPARATIONS 46 TO 58

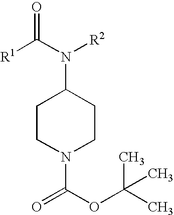

Oxalyl chloride (1.25-5 eq) was added dropwise to an ice-cooled solution of the acid (R¹CO₂H) (1 eq) and N,N-dimethylformamide (1 drop) in dichloromethane (2.5-6 ml/mmol), and the solution was allowed to warm to room temperature and stirred for 2 hours. The solution was concentrated under reduced pressure and the residue was azeotroped with toluene or dichloromethane (2×) to afford the intermediate acid chloride. This was re-dissolved in dichloromethane (3-5 ml/mmol), the appropriate Boc protected piperidine (1-1.5 eq) and triethylamine (2-4 eq) were added dropwise, and the reaction was stirred at room temperature for 18 hours. The mixture was washed with water, then brine, dried (MgSO₄) and concentrated under reduced pressure. The crude product was purified by column chromatography on silica gel using dichloromethane:methanol (100:0 to 95:5) as eluant to afford the title compounds.

| Prep. | | | | |
|---|---|---|---|---|
| no | Precursor | R¹ | R² | Data |
| 46 | Prep 39 | 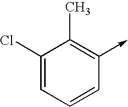 | 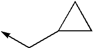 | ¹H-nmr (CDCl₃ 400 MHz) δ rotamers: −0.19, 0.41, 0.55 and 0.72 (4xm, 4H), 1.42, 1.47 (2xs, 9H), 1.74 (m, 5H), 2.28, 2.33 (2xs, 6H), 2.92 (m, 2H), 3.30 (m, 2H), 4.10, 4.26, 4.51 (3xm, 3H), 7.04 (m, 1H), 7.14 (m, 1H), 7.35 (m, 1H). LRMS: m/z APCI⁺ 407/409 [MH⁺] |
| 47 | Prep 32 | 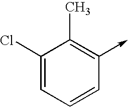 | 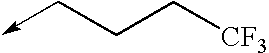 | LRMS: m/z APCI⁺ 485/487 [MNa⁺] |
| 48^{a} | Prep 37 | 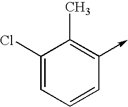 | 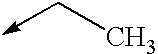 | ¹H-nmr (CDCl₃ , 400 MHz) δ rotamers: 0.95, 1.25 (2xt, 3H), 1.40, 1.45 (2xs, 9H), 1.50-1.85 (m, 4H), 2.25 (2xs, 3H), 2.70-3.60 (m, 4H), 4.00-4.30 (m, 2H), 7.00-7.40 (m, 3H). LRMS: m/z APCI⁺ 381 [MH⁺] |
| 49¹ | Prep 30 | 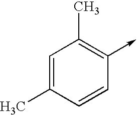 | 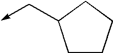 | ¹H-nmr (CD₃OD, 400 MHz) δ rotamers: 0.92 (br, 1H), 1.05 (br, 1H), 1.30-1.40 (br, 3H), 1.41-1.50 (m, 9H), 1.55-1.87 (m, 8H), 2.12 (m, 1H), 2.23 (d, 3H), 2.33 (d, 3H), 2.62 (br, 1H), 3.35-3.52 (m, 2H), 3.90-4.25 (m, 3H), 7.04-7.13 (m, 3H) LRMS: m/z APCI⁺ 415 [MH⁺] |
| 50¹ | Prep 30 | 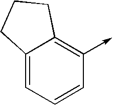 | 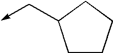 | ¹H-nmr (CD₃OD, 400 MHz) δ: 0.92-1.07 (br, 1H), 1.25-1.50 (br, 13H), 1.50-1.86 (br, 7H), 2.02-2.50 (br, 5H), 2.75-2.95 (br, 5H), 3.11-3.25 (br, 1H), 3.37-3.63 (br, 2H), 4.04-4.25 (br, 2H), 7.00-7.07 (br, 1H), 7.14-7.32 (br, 2H) LRMS: m/z APCI⁺ 427 [MH⁺] |
| 51 | Prep 36 | 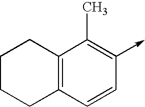 | 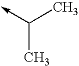 | ¹H-nmr (CDCl₃ , 400 MHz) δ: 1.10 (m, 7H), 1.43 (d, 9H), 2.15 (s, 3H), 2.60 (m, 2H), 2.75 (m, 2H), 2.80-3.20 (2m, 4H), 3.45 (m, 2H), 3.77 (m, 2H), 4.00-4.30 (2xs, 2H), 6.98-6.96 (2xt, 2H). LRMS m/z APCI⁺ 415 [MH⁺] |
| 52^{2b} | Prep 37 | 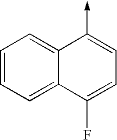 | 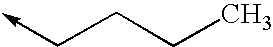 | ¹H-nmr (CD₃OD, 400 MHz) δ: 0.55 (t, 3H), 0.95 (m, 1H), 1.05 (t, 1H), 1.15 (s, 4.5H) 1.45 (s, 4.5H), 1.50-2.40 (br, 5H), 2.90 (m, 2H), 3.20-3.60 (br, 4H), 3.95-4.40 (br, SH), 7.25 (m, 1H), 7.40 (m, 1H), 7.65 (m, 2H), 7.80 (m, 1H), 8.20 (m, 1H). LRMS: m/z ES⁺ 451 [MNa]⁺ |
| 53¹ | Prep 29 | 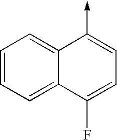 | 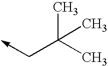 | ¹H-nmr (CDCl₃ , 400 MHz) 51.10 (s, 9H), 1.40 (s, 9H), 1.45 (m, 4H), 1.85-2.30 (3xbr, 2H), 2.60-3.50 (4xbr, 4H), 3.90-4.20 (2xbr, 1H), 7.1 (t, 1H), 7.30-7.40 (2xm, 1H), 7.55 (m, 2H), 7.75-8.05 (2xm, 1H), 8.15 (m, 1H) LRMS: m/z APCI⁺ 443 [MH⁺] |
| 54² | Prep 29 | 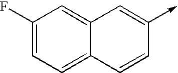 | 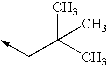 | ¹H-nmr (CD₃OD, 400 MHz): δ Rotamers: 0.70 (s, 4.5H), 1.10 (s, 4.5H), 1.35 (s, 4.5H), 1.45 (m, 4.5H), 1.60 (m, 0.5H), 1.70 (s, 0.5H), 1.90-2.40 (br, 2H), 2.80 (m, 2H), 3.00 (d, 0.5H), 3.25 (d, 0.5H), 3.45-3.60 (br, 2H), 7.17 (m, 1H), 7.40 (m, 0.5H), 7.55 (m, 0.5H), 7.65 (m, 2H), 7.80 (m, 0.5H), 8.0 (m, 0.5H), 8.20 (m, 1H) LRMS: m/z ES⁺ 465 [MNa⁺] |
| 55¹ | Prep 34 | 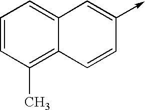 | 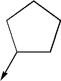 | ¹H-nmr (CDCl₃ , 400 MHz) δ rotamers: 1.45 (s, 9H), 1.10-1.50 (m, 4H), 1.70 (d, 2H), 1.75 (m, 4H), 1.90 (br, 2H), 2.50-2.60 (br, 2H), 2.70 (s, 3H), 3.40 (br, 1H), 3.80 (br, 1H), 4.2 (br, 2H), 7.35 (d, 1H), 7.45 (m, 2H), 7.70 (d, 1H), 7.80 (s, 1H), 8.00 (d, 1H) LRMS m/z APCI⁺ 337 [MH⁺ − BOC + H⁺], |
| 56^{1c} | Prep 38 | 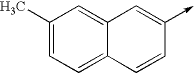 | 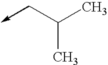 | ¹H-nmr (CDCl₃ , 400 MHz) δ rotamers: 0.60-1.15 (br, 6H), 1.45 (s, 9H), 1.45 (br, 1H), 1.60-2.00 (br, 4H), 2.10-2.60 (br, 2H), 2.55 (s, 3H), 3.20 (d, 2H), 3.80 (br, 1H), 4.15 (br, 2H), 7.35 (d, 2H), 7.65 (s, 1H), 7.75 (d, 2H), 7.80 (d, 1H) LRMS: m/z APCI⁺ 425 [MH⁺] |
| 57^{1,2} | Prep 34 | 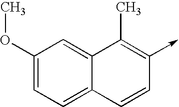 | 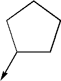 | ¹H-nmr (CD₃OD, 400 MHz): δ Rotamers: 1.20 (s, 4.5H), 1.25 (s, 4.5H), 1.60-1.90 (br, 4H), 2.00 (m, 1H), 2.40 (m, 1H), 2.62 (s, 3H) 2.85 (m, 2H), 3.80 (br, 2H) 3.90 (s, 3H), 4.05-4.10 (br, 2H), 7.05 (m, 1H), 7.10 (m, 1H), 7.35 (m, 1H), 7.70 (m, 1H), 7.80 (m, 1H) LRMS: m/z ES⁺ 467 [MH⁺] |
| 58 | Prep 34 | 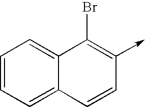 | 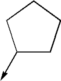 | ¹H-nmr (CDCl₃, 400 MHz) δ rotamers: 1.43, 1.49 (2xs, 9H), 1.52-4.30 (m, 18H), 7.22-7.26 (m, 1H), 7.54-7.60 (m, 1H), 7.62-7.67 (m, 1H), 7.83, 7.86 (2xm, 2H), 8.30 (dd, 1H). LRMS: m/z APCI⁺ 503/501 [MH⁺]. |

¹= ethyl acetate:pentane was used as the column eluant
²= 1.5 eq of Hunig's base was used instead of triethylamine
^{a}= 1-(tert-butoxycarbonyl)-4-(ethylamino)piperidine (US 0094989, pg 54) was used as the starting amine.
^{b}= 1-(tert-butoxycarbonyl)-4-(butylamino)piperidine (WO 09618628, ex 86) was used as the starting amine.
^{c}= 1-(tert-butoxycarbonyl)-4-(isobutylamino)piperidine (WO 09618628, ex 90) was used as the starting amine.

### PREPARATIONS 59 TO 69

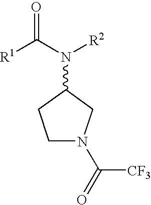

The compounds of the general formula shown above were prepared from the appropriate acid (R¹CO₂H) and protected pyrrolidine, following the procedure described for preparations 46 to 58.

| Prep. | | | | | |
|---|---|---|---|---|---|
| no | Stereo | Precursor | R¹ | R² | Data |
| 59^{a} | R | Prep 21 | 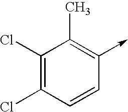 | 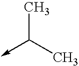 | LRMS: m/z APCI⁺ 411/413 [MH⁺] |
| 60 | R | Prep 22 | 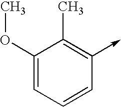 | 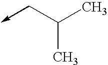 | ¹H-nmr (CD₃OD, 400 MHz) δ: 0.77 (m, 3H), 1.01 (m, 3H), 1.85 (m, 1H), 2.08-2.20 (m, 3H), 2.21-2.45 (m, 2H), 2.95 (t, 1H), 3.12-3.34 (m, 2H), 3.39-3.79 (m, 3H), 3.96 (m, 1H), 6.77 (d, 1H), 6.97 (m, 1H), 7.20-7.60 (m, 1H) LRMS: m/z APCI⁺ 387 [MH⁺] |
| 61 | R | Prep 22 | 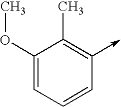 | 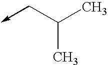 | ¹H-nmr (CD₃OD, 400 MHz) δ: 0.75 (brd, 6H), 1.75-2.00 (m, 1H), 2.10-2.30 (m, 1H), 2.40 (s, 3H), 3.13 (d, 2H), 3.40-4.05 (m, 5H), 4.30-4.45 (m, 1H), 7.22 (d, 1H), 7.35 -7.44 (m, 2H) LRMS: m/z APCI⁺ 391 [MH⁺] |
| 62¹ | R | Prep 22 | 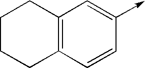 | 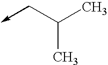 | LRMS: m/z APCI⁺ 397 [MH⁺] |
| 63 | S | Prep 28 | 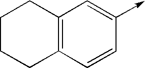 | 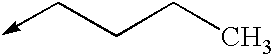 | LRMS: m/z APCI⁺ 397 [MH⁺] |
| 64 | R | Prep 25 | 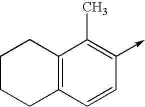 | 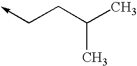 | ¹H-nmr (CDCl₃, 400 MHz) δ: 0.65 (s, 3H), 0.98 (s, 3H), 1.25 (t, 2H), 1.20-1.90 (brm, 8H), 2.10 (s, 4H), 2.60 (s, 2H), 2.80 (t, 2H), 3.10 (d, 2H), 3.15-4.10 (brm, 4H), 6.80-7.00 (m, 2H), LRMS m/z APCI⁺ 425 [MH⁺] |
| 65 | R | Prep 24 | 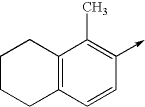 | 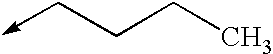 | ¹H-nmr (CDCl₃ , 400 MHz) δ: 0.71 (t, 3H), 0.95-1.10 (m, 2H), 1.35 (d, 2H), 1.70-1.90 (m, 4H), 2.10 (3H), 2.60 (s, 2H), 2.76 (s, 2H), 3.00-4.80 (m, 7H), 6.81-6.89 (m, 2H). LRMS: m/z APCI⁺ 411 [MH⁺ |
| 66 | S | Prep 23 | 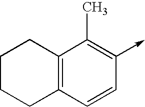 | 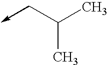 | ¹H-nmr (CDCl₃, 400 MHz) δ: 0.64 (s, 3H), 1.00 (s, 3H), 1.85 (m, 6H), 2.50-2.10 (m, 4H), 2.60 (s, 3H), 2.75 (s, 2H), 3.10-3.85 (m, 4H), 4.00 (m, 1H), 4.25 (m, 1H), 6.83-7.00 (m, 2H). LRMS: m/z APCI⁺ 411 [MH⁺] |
| 67² | R | Prep 24 | 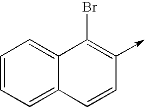 | 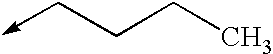 | ¹H-nmr (CDCl₃, 400 MHz) δ: 0.70-1.05 (2xt, 3H), 1.25-1.55 (2xm, 2H), 1.60-2.05 (m, 2H), 2.05-2.65 (m, 2H), 3.05-4.30 (m, 7H), 4.60-4.85 (2xm, 1H), 7.25-7.45 (m, 1H), 7.50-7.70 (m, 2H), 7.80-7.95 (m, 2H), 8.30 (d, 1H) LRMS m/z (APCI⁺) 473 [MH⁺] |
| 68 | S | Prep 23 | 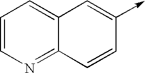 | 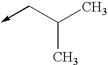 | LRMS: m/z APCI⁺ 394 [MH⁺] |
| 69 | R | Prep 22 | 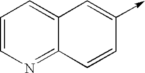 | 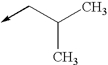 | ¹H-nmr (CDCl₃ , 400 MHz) δ: 0.83 (s, 6H), 1.82-2.64 (brm, 3H), 3.24 (m, 2H), 3.42-4.11 (brm, 4H), 4.38 (m, 1H), 7.49 (m, 1H), 7.68 (d, 1H), 7.86 (s, 1H), 8.20 (m, 2H), 8.99 (s, 1H) LRMS: m/z APCI⁺ 394 [MH⁺] |

¹= DMAP (catalytic) was also added
²= pentane:ethyl acetate was used as the column eluant
^{a}= 3,4-dichloro-2-methylbenzoic acid (J. Med. Chem. 1997; 40(13); 2017) was used as the starting acid.

### PREPARATION 70

### N-Butyl-N-[(3R)-1-(trifluoroacetyl)pyrrolidin-3-yl]-2-naphthamide

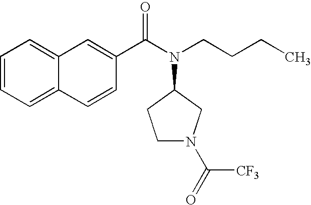

A mixture of 2-naphthoyl chloride (240 mg, 1.26 mmol), the pyrrolidine from preparation 24 (200 mg, 0.84 mmol) and triethylamine (350l, 2.5 mmol) in dichloromethane (5 ml) was stirred at room temperature for 18 hours. The mixture was partitioned between ethyl acetate (50 ml) and sodium bicarbonate solution (50 ml), the layers were separated, and the organic phase was dried (MgSO₄) and evaporated under reduced pressure. The crude product was purified by column chromatography on silica gel using an elution gradient of pentane:ethyl acetate (90:10 to 70:30) to afford the title compound, 320 mg.

¹H -nmr (CDCl₃, 400 MHz) δ 0.95 (t, 3H), 1.25 (m, 2H), 1.60 (m, 2H), 1.60-2.30 (2×m, 2H), 3.30 (m, 2H), 3.40-3.80 (m, 2H), 3.85-4.00 (2×m, 2H), 4.45(2×m, 1H), 7.45 (d, 1H), 7.50-7.60 (m, 2H), 7.85-7.95 (m, 4H)

LRMS: m/z APCI⁺ 393 [MH⁺]

### PREPARATION 71

### N-Benzyl-N-[(3R)-1-(trifluoroacetyl)pyrrolidin-3-yl]-2-naphthamide

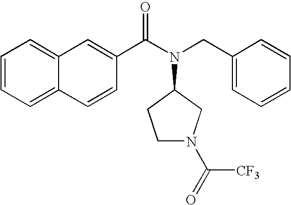

The title compound was obtained in 58% yield from the pyrrolidine from preparation 26 and 2-naphthoyl chloride, following a similar procedure to that described in preparation 67, except that 0.1 eq of DMAP was added to the reaction mixture.

¹H -nmr (CDCl₃, 400 MHz) δ: 2.05-2.40 (m, 2H), 3.35-3.75 (m, 2H), 3.75-4.00 (m, 2H), 4.50-4.70 (m, 1H), 4.50-4.80 (m, 2H), 7.20-7.35 (m, 5H), 7.45-7.60 (m, 3H), 7.80-7.90 (m, 3H), 7.90-7.95 (d, 1H)

LRMS: m/z APCI⁺ 427 [MH⁺]

### PREPARATION 72

### N-(2-Methylbutyl)-N-[(3R)-1-(trifluoroacetyl)pyrrolidin-3-yl]-2-naphthamide

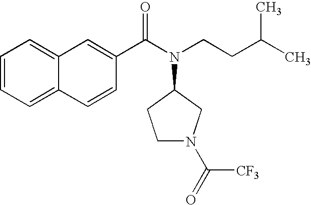

A mixture of 2-naphthoyl chloride (150 mg, 0.79 mmol), the pyrrolidine from preparation 25 (198 mg, 0.79 mmol) and triethylamine (0.22 ml, 1.57 mmol) in dichloromethane (5 ml) was stirred at room temperature for 18 hours. The reaction was washed with water and brine, then dried (MgSO₄) and evaporated under reduced pressure. The residue was purified by column chromatography on silica gel using an elution gradient of dichloromethane:methanol (100:0 to 96:4) to afford the title compound as an oil, 230 mg.

¹H-nmr (CDCl₃ 400MHz) δ: 0.8 (br, 6H), 1.5 (br, 2H), 2.65 (m, 2H), 3.3-3.5 (m, 3H), 3.5-4.0 (m, 4H), 4.5-4.7 (m, 2H), 7.4 (d,1H), 7.5-7.6 (m, 2H), 7.8-7.9 (m, 4H)

LRMS: m/z APCI⁺ 407 [MH⁺]

### PREPARATION 73

### N-Isobutyl-N-[(3R)-1-(trifluoroacetyl)pyrrolidin-3-yl1-2-naphthamide

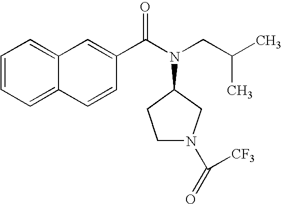

The title compound was obtained as an oil in 53% yield, from 2-naphthoyl chloride and the pyrrolidine from preparation 22, following a similar procedure to that described in preparation 72 above.

¹H-nmr (CDCl₃, 400 MHz) δ: 0.86 (s, 6H), 1.98 (br, 1H), 2.17-2.60 (brm, 2H), 3.24 (m, 2H), 3.40-4.06 (brm, 4H), 4.40 (m, 1H), 7.44 (d, 1H), 7.58 (m, 2H), 7.89 (m, 4H)

### PREPARATION 74

### Methyl 2-({naphthoyl[(3R)-1-(trifluoroacetyl)pyrrolidin-3-yl]aminolmethyl)benzoate

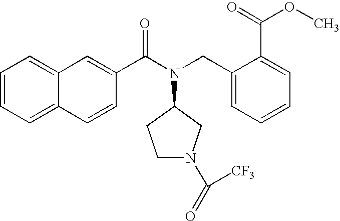

The title compound was obtained as an oil, from 2-naphthoyl chloride and the pyrrolidine from preparation 27 following the procedure described in preparation 72. ¹H-nmr (CDCl₃ 400 MHz) δ: 2.2-2.5 (m, 2H), 3.6-3.8 (m, 4H), 3.9-4.1 (m, 3H), 4.4 (s, 2H), 5.1 (m, 1H), 7.4-7.6 (m, 7H), 7.8-7.9 (m, 4H)

LRMS m/z APCI⁺ 485 [MH⁺]

### PREPARATION 75

### tert-Butyl 4-[cyclopentyl(1-methyl-2-naphthoyl)amino]piperidine-1-carboxylate

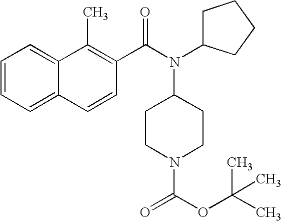

A mixture of the compound from preparation 58 (200 mg, 0.4 mmol), trimethyl boroxine (100 mg, 0.8 mmol), tetrakis(triphenylphosphine)palladium (0) (46 mg, 0.04 mmol) and potassium carbonate (221 mg, 1.6 mmol) in dioxan (5 ml) was heated to 90° C. Once this temperature had been reached, water (3 drops) was added and the reaction was stirred at 90° C. for 18 hours. The cooled mixture was evaporated under reduced pressure and the residue was purified by column chromatography on silica gel using a Varian Bond Elut® Cartridge and an elution gradient of pentane:ethyl acetate (100:0 to 50:50) to afford the title compound as a white solid, 156 mg.

¹H-nmr (CDCl₃, 400 MHz) δ rotamers: 1.43,1.49 (2×s, 9H), 1.51-4.27 (m, 21H), 7.20 (t, 1H), 7.49-7.59 (m, 2H), 7.72 (d, 1H), 7.83-7.85 (m, 1H), 8.04 (d, 1H).

LRMS: m/z APCI⁺ 437 [MH⁺]

### PREPARATION 76

### tert-Butyl 4-{(naphthalene-2-carbonyl)-[4-(naphthalene-2-carbonyloxy)-butyl]-amino}piperidine-1-carboxylate

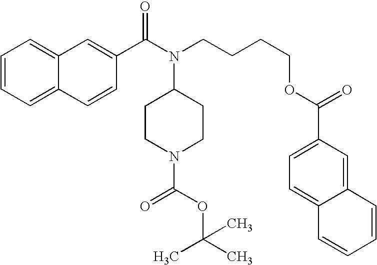

A solution of the alcohol from preparation 31 (500 mg, 1.84 mmol) and triethylamine (768 μl, 5.51 mmol) in dichloromethane (10 ml) was cooled in an ice-bath. 2-Naphthoyl chloride (875 mg, 4.59 mmol) was added portionwise and the reaction mixture was stirred at room temperature for 18 hours. The mixture was partitioned between dichloromethane (50 ml) and water (50 ml), and the layers were separated. The organic phase was dried (MgSO₄), and evaporated under reduced pressure. The product was purified by chromatography on silica gel using a Varian Bond Elut® cartridge, and an elution gradient of dichloromethane:methanol:0.88 ammonia (100:0:0 to 95:5:0.5) to afford the title compound as a pink oil, 1.02 g.

LRMS: m/z APCI⁺ 581 [MH⁺]

### PREPARATION 77

### tert-Butyl 4-[(4-hydroxy-butyl)-(naphthalene-2-carbonyl)-aminol]piperidine-1-carboxylate

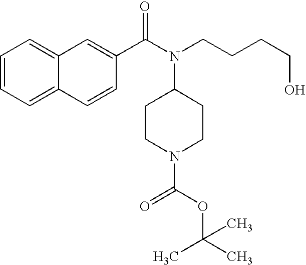

Sodium methoxide (8.5 mg, 0.26 mmol) was added to a solution of the compound from preparation 76 (1.02 g, 1.76 mmol) in methanol (17 ml) and the reaction stirred at room temperature for 18 hours. The mixture was partitioned between water (100 ml) and ethyl acetate (100 ml), and the layers were separated. The organic phase was dried (MgSO₄) and evaporated under reduced pressure. The crude product was purified by chromatography on silica gel using a Varian Bond Elut® cartridge, and an elution gradient of dichloromethane:methanol:0.88 ammonia (100:0:0 to 90:10:1) to afford the title compound as a yellow oil, 780 mg.

¹H-nmr (CDCl₃, 400 MHz) δ: 1.15-1.90 (brm, 9H), 1.43 (s, 9H), 2.41 (br, 1H), 3.40 (br, 2H), 3.73 (br, 2H), 4.16 (br, 2H), 7.42 (d, 1H), 7.55 (m, 2H), 7.80-7.92 (m, 4H)

LRMS: m/z APCI⁺ 427 [MH⁺]

### PREPARATION 78

### 1-Chloro-N-butyl-N-[(3R)-1-(trifluoroacetyl)pyrrolidin-3-yl]-2-naphthamide

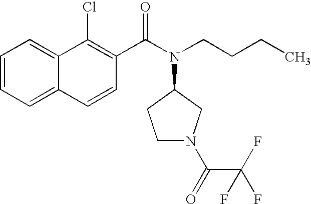

A mixture of the bromide from preparation 67 (300 mg, 0.63 mmol), and copper chloride (740 mg, 7.48 mmol) in dimethyl sulphoxide (6 ml) was heated at 120° C. for 18 hours, then allowed to cool-to room temperature. The mixture was diluted with ethyl acetate, filtered through Celite®, and the filtrate was washed with water (80 ml), then brine (80 ml). The organic solution was dried (MgSO₄) and evaporated under reduced pressure. The crude product was purified by column chromatography on silica gel using ethyl acetate:pentane (50:50) as eluant to afford the title compound as a yellow oil, 280 mg.

¹H-nmr (CDCl₃, 400 MHz) δ: 0.70-1.05 (2×t, 3H), 1.30-1.55 (m, 2H), 1.60-2.05 (m, 2H), 2.05-2.65 (m, 2H), 3.05-4.30 (m, 7H), 4.60-4.80 (m, 1H), 7.25-7.35 (m, 1H), 7.40-7.70 (m, 2H), 7.80-7.95 (m, 2H), 8.35 (d, 1H)

LRMS: m/z ES+427 [MH⁺]

1. A Compound of formula (I), 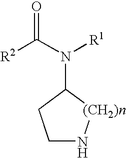
wherein R¹ is selected from:
(a) (C₁-C₆)alkyl, optionally substituted by 1-3 substituents, each independently selected from:
(i) CF₃, OH, (C₁-C₆)alkyl, (C₃-C₆)cycloalkyl, (C₁-C₆)alkoxy and halo;
(ii) Phenyl, optionally fused with phenyl or cyclohexyl, said phenyl or fused phenyl optionally substituted with 1-3 groups selected from (C₁-C₆)alkyl, (C₁-C₆)alkyl ester, OH and halo; and
(b) (C₃-C₆)cycloalkyl, optionally fused with (C₅-C₇)cycloalkyl, said cycloalkyl or fused cycloalkyl optionally substituted by OH, (C₁-C₆)alkyl, (C₁-C₆)alkoxy and halo;
R² is Phenyl, optionally fused to (C₄-C₆)cycloalkyl, phenyl or pyridyl, said phenyl or fused phenyl moiety optionally substituted with 1-3 groups each independently selected from (C₁-C₆)alkyl, (C₁-C₆)alkoxy, halo and OH;
n is 1 or 2;
and pharmaceutically acceptable salts, solvates or polymorphs thereof;
With the proviso that when n is 2 and R¹ is 2-(3,4-dimethoxylphenyl)-1-ethyl, 3,3-diphenyl-1-propyl or 2,4-difluorophenyl, then R² cannot be 4-trifluoromethoxyphenyl, 2,4,6-trimethoxyphenyl, 4-acetoxyphenyl or 2,4-difluorophenyl.

2. The Compound of formula (I), as claimed in claim 1, wherein R1 is selected from:
(a) (C₁-C₅)alkyl, optionally substituted by 1-2 substituents, each independently selected from:
(i) OH, (C₃-C₄)cycloalkyl; or
(ii) Phenyl optionally substituted by C(O)OCH₃; or
(b) (C₄-C₅)cycloalkyl

3. The Compound of formula (I), as claimed in claim 1, wherein R¹ is selected from:
(C₃-C₅)alkyl; (C₁)alkyl substituted by phenyl, cyclobutyl or cyclopropyl; and (C₄-C₅)cycloalkyl.

4. Compounds of formula (I), as claimed in claims 1 to 3, wherein R² is phenyl, optionally fused to cyclohexyl, phenyl or pyridyl, said phenyl or fused phenyl moiety optionally substituted with 1-3 groups each independently selected from methoxy, methyl, chloro and fluoro.

5. The Compound of Formula (I) of claim 4, wherein n is 1.

6. The Compound of formula (I), as claimed in claims 1 to 3, wherein R² is phenyl, optionally substituted with 2-3 groups each independently selected from methoxy, methyl and chloro; or phenyl fused to cyclohexyl, phenyl or pyridyl and optionally substituted with 1-2 groups independently selected from methyl, methoxy and chloro.

7. The Compound of Formula (I) of claim 6, wherein n is 1.

8. The Compound of formula (I), as claimed in claims 1 to 3, wherein n is 1.

9. A compound selected from:
N-Cyclopentyl-N-piperidin-4-yl-2-naphthamide;
N-Butyl-3-chloro-2-methyl-N-piperidin-4-ylbenzamide;
3-Chloro-N-(cyclopropylmethyl)-2-methyl-N-piperidin-4-ylbenzamide;
N-Isobutyl-N-[(3R)-pyrrolidin-3-yl]-2-naphthamide;
N-Isobutyl-N-[(3R)-pyrrolidin-3-yl]quinoline-6-carboxamide;
N-Isobutyl-N-[(3S)-pyrrolidin-3-yl]quinoline-6-carboxamide;
N-(3-Methylbutyl)-N-[(3R)-pyrrolidin-3-yl]-2-naphthamide;
3,4-Dichloro-N-isopropyl-2-methyl-N-[(3R)-pyrrolidin-3-yl]benzamide;
N-Butyl-1-methyl-N-[(3R)-pyrrolidin-3-yl]-5,6,7,8-tetrahydronaphthalene-2-carboxamide;
N-Butyl-N-[(3R)-pyrrolidin-3-yl]-2-naphthamide;
N-Butyl-1-chloro-N-[(3R)-pyrrolidin-3-yl]-2-naphthamide;
N-Isobutyl-3-methoxy-2-methyl-N-[(3R)-pyrrolidin-3-yl]benzamide;
and pharmaceutically acceptable salts, solvates or polymorphs thereof.

10. A method of treatment or prevention of depression, attention deficit hyperactivity disorder, obsessive-compulsive disorder, post-traumatic stress disorder, substance abuse disorders or sexual dysfunction including premature ejaculation, which comprises administering a therapeutically effective amount of the compound of formula (I), as claimed in claims 1-3 and 9, to a patient in need of such treatment or prevention.

11. The method of treatment according to claim 10 wherein R² is phenyl, optionally fused to cyclohexyl, phenyl or pyridyl, the phenyl or fused phenyl moiety optionally substituted with 1-3 groups each independently selected from methoxy, methyl, chloro and fluoro.

12. The method of treatment according to claim 10 wherein R² is phenyl, optionally substituted with 2-3 groups each independently selected from methoxy, methyl and chloro; or phenyl fused to cyclohexyl, phenyl or pyridyl and optionally substituted with 1-2 groups independently selected from methyl, methoxy and chloro.

13. A method of increasing ejaculatory latency which comprises the administration of a therapeutically effective amount of the compound of formula (I), as claimed in claims 1-3 and 9, to a male desiring increased ejaculatory latency.

14. A method according to claim 13 wherein R² is phenyl, optionally fused to cyclohexyl, phenyl or pyridyl, the phenyl or fused phenyl moiety optionally substituted with 1-3 groups each independently selected from methoxy, methyl, chloro and fluoro.

15. A method according to claim 13 wherein R² is phenyl, optionally substituted with 2-3 groups each independently selected from methoxy, methyl and chloro; or phenyl fused to cyclohexyl, phenyl or pyridyl and optionally substituted with 1-2 groups independently selected from methyl, methoxy and chloro.

16. A pharmaceutical composition including a compound of formula (I), as claimed in claims 1-3 and 9, and a pharmaceutically acceptable diluent or carrier.

17. A pharmaceutical combination including a compound of formula (I), as claimed in claims 1-3 and 9, and another pharmacologically active agent.
